(19) United States
(12) Patent Application Publication
(10) Pub. No.: US 2024/0325520 A1
(43) Pub. Date: Oct. 3, 2024

(54) Recombinant Severe Acute Respiratory Syndrome Coronavirus 2 Rbd Trimer Protein Vaccine Capable Of Generating Broad-Spectrum Cross Neutralization Activity, and Preparation Method and Use Thereof

(71) Applicant: NATIONAL VACCINE AND SERUM INSTITUTE(NVSI), Daxing District, Beijing (CN)

(72) Inventors: Qiming LI, Daxing District, Beijing (CN); Yu LIANG, Daxing District, Beijing (CN); Jing ZHANG, Daxing District, Beijing (CN); Jiguo SU, Daxing District, Beijing (CN); Zibo HAN, Daxing District, Beijing (CN); Shuai SHAO, Daxing District, Beijing (CN); Yanan HOU, Daxing District, Beijing (CN); Hao ZHANG, Daxing District, Beijing (CN); Shi CHEN, Daxing District, Beijing (CN); Yuqin JIN, Daxing District, Beijing (CN); Xuefeng ZHANG, Daxing District, Beijing (CN); Lifang DU, Daxing District, Beijing (CN); JunWei HOU, Daxing District, Beijing (CN); Zhijing MA, Daxing District, Beijing (CN); Zehua LEI, Daxing District, Beijing (CN); Fan ZHENG, Daxing District, Beijing (CN); Fang TANG, Daxing District, Beijing (CN); Zhaoming LIU, Daxing District, Beijing (CN); Ning LIU, Daxing District, Beijing (CN)

(21) Appl. No.: 18/277,087

(22) PCT Filed: Sep. 24, 2021
(86) PCT No.: PCT/CN2021/120447

§ 371 Date: Aug. 14, 2023

(30) Foreign Application Priority Data
Jun. 18, 2021 (CN) 202110676901.2

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/55505; A61K 2039/575; A61K 2039/70; C12N 2770/20022; C12N 2770/20034

(57) ABSTRACT

The present invention discloses a recombinant RBD trimer protein capable of simultaneously generating cross neutralization activity for various severe acute respiratory syndrome coronavirus 2 (SARS-CoV-2) epidemic strains. The RBD trimer protein is taken as an antigen and supplemented with an adjuvant to immunize an organism, so that a high-titer neutralizing antibody aiming at various SARS-CoV-2 epidemic strains can be generated at the same time, and the antibody has a certain broad-spectrum property and can be used for treating and/or preventing SARS-CoV-2 infection and/or coronavirus disease 2019.

### CROSS-REFERENCE

This application claims the priority of a Chinese patent application entitled “RECOMBINANT SEVERE ACUTE RESPIRATORY SYNDROME CORONAVIRUS 2 RBD TRIMER PROTEIN VACCINE CAPABLE OF GENERATING BROAD-SPECTRUM CROSS NEUTRALIZATION ACTIVITY, AND PREPARATION METHOD AND USE THEREOF” submitted to the China Patent Office on Jun. 18, 2021, with application No. 202110676901.2, and the entire content of which is incorporated in this application by reference.

### TECHNICAL FIELD

The present invention relates to the field of biological drugs, and in particular to a recombinant SARS-CoV-2 RBD trimer protein vaccine capable of generating broad-spectrum cross neutralization activity, and a preparation method and use thereof.

### BACKGROUND

Severe acute respiratory syndrome coronavirus 2 (SARS-CoV-2) belongs to a single-stranded positive strand RNA virus, SARS-like virus species, sarbecovirus subgenus, betacoronavirus, positive coronavirus, coronaviridae, nidovirales, with a cyst membrane. The full length of genome is about 29.9 kb. Most of the genome encodes non-structural proteins and participates in virus replication and translation functions; and a few sequences encode structural proteins, such as a spike protein (S protein), a membrane protein (M protein), a cyst membrane protein (E protein) and a nucleo protein (N protein). In addition, there are several accessory proteins: 3a, 3b, p6, 7a, 7b, 8b, 9b and or f14, and these proteins participate in viral assembly. S, M and E proteins constitute a virus cyst membrane, which is a main surface antigen of the virus causing immune response. The S protein is a transmembrane glycoprotein, has a molecular weight about 150 kDa, and forms a prominent homotrimer on the surface of the virus. The S protein consists of two functional subunits, and is cleaved at a boundary (an S1/S2 cleavage site) between the S1 subunit and the S2 subunit; and the two subunits keep non-covalent binding in conformation before fusion. The S2 subunit is composed of a plurality of structural domains, and has a main function of mediating the fusion between a virus and a host cell. The distal S1 subunit is structurally divided into four different structural domains: a N-terminal structural domain (NTD), a receptor binding structural domain (RBD), a C-terminal structural domain 1 (CTD1) and a C-terminal structural domain 2 (CTD2), where RBD is mainly responsible for binding with a receptor angiotensin converting enzyme 2 (ACE2) on the surface of the host cell, so that the virus is mediated to infect the host cell; therefore, the S protein and RBD are the main targets of the research and development of genetic engineering vaccine at present.

Up to now, there are totally eight vaccines approved for listing in the world, namely BNT162b2 and mRNA-1273 approved by the United States for emergency use authorization (EUA), AZD1222 approved by the United Kingdom for EUA, three types of COVID-19 inactivated vaccines from China National Biotec Group (Beijing company and Wuhan company) and Beijing Sinovac Biotech Ltd., CanSinoBIO adenovirus vector vaccines and Zhifei Biological recombinant protein vaccines, and “satellite V” approved by Russia for listing. In addition, there are dozens of vaccines in different stages of clinical research. No matter which technical route these listed vaccines come from, different contributes are made to epidemic prevention and control. However, due to the evolution of SARS-CoV-2, various mutations occur continuously, so that the protective effect of existing vaccines is affected to varying degrees.

SARS-CoV-2 belongs to an RNA virus, which is prone to mutation. More than 30,000 SARS-CoV-2 mutant strains have been reported in the world, mainly including: an alpha (B.1.1.7) mutant strain, a beta (B.1.351) mutant strain, a gamma (P1) mutant strain, an epsilon (B.1.429) mutant strain, a delta (B.1.617.2) mutant strain, and a kappa (B.1.617.1) mutant strain. The mutation sites of these mutant strains are mainly present in the amino acid sequences of the S protein, in particular an RBD region. Therefore, mutation may improve the affinity between the virus and an ACE2 receptor and attenuating neutralizing antibody effect, so that the virulence and infectivity of the virus are enhanced, virus escape is accelerated, and the protective effect of the vaccines is reduced. Therefore, it is urgent to develop a broad-spectrum vaccine against various SARS-CoV-2 epidemic strains.

### SUMMARY

For the technical defect of lacking a broad-spectrum severe acute respiratory syndrome coronavirus 2 (SARS-CoV-2) vaccine in the prior art, the present invention provides a single-component broad-spectrum RBD trimer protein vaccine capable of generating high cross neutralization activity for various SARS-CoV-2 epidemic strains.

The technical solution provided by the present invention is as follows:
a recombinant SARS-CoV-2 RBD trimer protein capable of generating broad-spectrum cross neutralization activity, where the trimer protein is composed of subunits of three SARS-CoV-2 RBD regions, and the amino acid sequences of the three SARS-CoV-2 RBD regions are the same or at least one of the three is different; and
when the amino acid sequences of the three SARS-CoV-2 RBD regions are the same, the amino acid sequences are the amino acid sequences shown in SEQ ID NO.2 or SEQ ID NO.3, or sequences with more than 95% of homology with the amino acid sequences shown in SEQ ID NO.2 or SEQ ID NO.3.

According to the present invention, a brand-new fusion protein is designed by a computational biology method based on the structural feature of a SARS-CoV-2 RBD region. The protein includes three RBD structural domains, which can form a trimer form with stable antigen conformation without introducing any exogenous linking arm or other irrelevant components, thereby realizing RBD protein trimerization. After recombinant expression and purification of an RBD trimer protein by a genetic engineering technology, the protein and an adjuvant are mixed to prepare a vaccine. After immunization with a certain dosage and times, a protective neutralizing antibody for various SARS-CoV-2 epidemic strains can be generated for treating and/or preventing SARS-CoV-2 infection and/or coronavirus disease 2019 (COVID-19). The RBD region has a clear function and structure and is responsible for identifying an ACE2 receptor of a host cell, and the antibody generated for the RBD has a clear function and a specific target, thereby preventing from inducing an organism to produce antibody dependent enhancement (ADE).

The trimer protein in the present invention is composed of amino acid fragments of three SARS-CoV-2 RBD regions that are connected in an order from an N terminal to a C terminal.

In the present invention, a length of peptide chains of three RBD region subunits of the trimer protein may be a full length of the SARS-CoV-2 S protein RBD region. Preferably, in the embodiments of the present invention, the length of the peptide length of the SARS-CoV-2 RBD region is at least 50% to 99% of the full length of the RBD region sequence.

50% to 99% of the full length of the RBD region may be 50%, 60%, 70%, 80%, 90% or 99% of the full length of the RBD region, and at least includes one or more different amino acid residues.

More preferably, in one embodiment of the present invention, the amino acid sequences of the SARS-CoV-2 RBD region are the 319th to 537th amino acids of the S protein.

In the present invention, the amino acid sequences of the SARS-CoV-2 RBD region may be from RBD structural domains of S protein of different SARS-CoV-2 epidemic strains, for example, the prototype strain SARS-CoV-2, Kappa (B.1.617) SARS-CoV-2 carrying L452R and E484Q mutants, SARS-CoV-2 carrying a D614G mutant, SARS-CoV-2 carrying an L452R mutant. Alpha (B.1.1.7) SARS-CoV-2 carrying a plurality of mutants N501Y, P681H and 69-70del, Beta (B.1.135) SARS-CoV-2 carrying a plurality of mutants N501Y, K417N and E484K, and Gamma (P.1) SARS-CoV-2 carrying N501Y, E484K and K417T mutants. The above sequences may be obtained through NCBI (https://www.ncbi.nlm.nih.gov/).

Preferably, in the embodiments of the present invention, the amino acid sequences of the SARS-CoV-2 RBD region are amino acid sequences shown in SEQ ID NO.1, SEQ ID NO.2 or SEQ ID NO.3, or sequences with more than 95% of homology with the amino acid sequences shown in SEQ ID NO.1, SEQ ID NO.2 or SEQ ID NO.3.

In the above embodiments, the trimer protein provided by the present invention may form a trimer form with stable antigen conformation without introducing any exogenous linking arm or other irrelevant components.

In the above embodiments, the amino acid sequences shown in SEQ ID NO.1, SEQ ID NO.2 or SEQ ID NO.3 may obtain new amino acid sequences by substituting, deleting or inserting one or more amino acid sequences. A new protein formed by the amino acid sequences has the same or basically the same immunological activity as the protein composed of the amino acid sequences shown in SEQ ID NO.1, SEQ ID NO.2 or SEQ ID NO.3. The new amino acid sequences may be considered to be included in the protection scope of the present invention.

Then, the sequences with more than 95% of homology with the amino acid sequences refer to amino acid sequences having 95%, 96%, 97%, 98% or 99% identity to the amino acid sequences of the recombinant SARS-CoV-2 RBD trimer protein or the fusion protein. Those skilled in the art can perform random or engineered point mutation on the amino acid sequences of the fusion protein in the specification, with the purpose of, for example, obtaining higher affinity and/or dissociation property and improving the expression performance. These sequences may have the same or basically the same immunological activity as the SARS-CoV-2 RBD trimer protein or the fusion protein, and these mutated amino acid sequences are all included in the protection scope of the present invention.

In the present invention, the three SARS-CoV-2 RBD regions with the same or at least one different amino acid sequence may be connected in any order from an N terminal to a C terminal. For example, in the embodiments of the present invention, the amino acid sequences of the protein formed by sequentially connecting SEQ ID NO.1, SEQ ID NO.2 and SEQ ID NO.3 is shown in SEQ ID NO.4; the amino acid sequences of the protein formed by sequentially connecting SEQ ID NO.1, SEQ ID NO.2 and SEQ ID NO.1 is shown in SEQ ID NO.5; the amino acid sequences of the protein formed by sequentially connecting SEQ ID NO.1. SEQ ID NO.2 and SEQ ID NO.2 is shown in SEQ ID NO.6; the amino acid sequences of the protein formed by sequentially connecting SEQ ID NO.2, SEQ ID NO.1 and SEQ ID NO.2 is shown in SEQ ID NO.7; and the amino acid sequences of the protein formed by sequentially connecting SEQ ID NO.2, SEQ ID NO.2 and SEQ ID NO.2 is shown in SEQ ID NO.8, or any other combination.

Preferably, in one embodiment of the present invention, the amino acid sequences of the trimer protein is amino acid sequences shown in SEQ ID NO.4, or sequences with more than 95% of homology with the amino acid sequences shown in SEQ ID NO.4.

Another aspect of the present invention provides a fusion protein. The fusion protein includes the recombinant SARS-CoV-2 RBD trimer protein.

Preferably, in the embodiments of the present invention, the fusion protein further includes one or more of signal peptides, tags or immune-enhancing peptides. The signal peptides may be more favorable for the expression of protein; and the tags may be, for example, flag tags, enhanced green fluorescence protein (eGFP), glutathione S-transferase (GST) and the like, and may be used for detection, purification, separation and the like. The above functional sequences may be used in any combination.

Another aspect of the present invention provides a nucleic acid molecule. The nucleic acid molecule includes a nucleotide sequence encoding the recombinant SARS-CoV-2 RBD trimer protein or encoding the fusion protein.

Preferably, in one embodiment of the present invention, the inventor optimizes a codon of the trimer protein, and the obtained nucleotide sequence is shown in SEQ ID NO.9 to SEQ ID NO.17 or sequences with more than 95% of homology with the amino acid sequences shown in SEQ ID NO.9 to SEQ ID NO.17.

The sequences with more than 95% of homology with the amino acid sequences refer to nucleotide sequences which are 95%, 96%, 97%, 98% or 99% the same as the nucleotide sequences.

For a method for preparing the nucleic acid molecule, the nucleic acid molecule may be prepared based on the above nucleotide sequences through known technologies such as chemical synthesis or PCR amplification. Generally, the codon of the amino acid encoding the above structural domain may be optimized to optimize the expression in a host cell. The information of the base sequence may be obtained by retrieving a database such as a known literature or NCBI (https://www.ncbi.nlm.nih.gov/).

Another aspect of the present invention provides a vector. The vector includes the nucleic acid molecule.

In the present invention, the vector may be a linear vector or a circular vector. The vector may be a non-viral vector such as plasmid, or a virus vector, or a vector using a transposon. The vector may include a promoter, a terminator or other regulatory sequences, and a drug resistance gene, a reporter gene or other marker sequence.

Preferably, in one embodiment of the present invention, the vector is an expression vector of the nucleic acid molecule in the present invention.

Another aspect of the present invention provides a host cell. The host cell includes the nucleic acid molecule or the vector.

Preferably, in the embodiments of the present invention, the host cell is Escherichia coli, a yeast cell, an insect cell or a mammalian cell.

Preferably, in one embodiment of the present invention, the host cell is a CHO cell.

Another aspect of the present invention provides a method for preparing the recombinant SARS-CoV-2 RBD trimer protein or the fusion protein, including the following steps:
step A): preparing the nucleic acid molecule, constructing an expression vector of the nucleic acid molecule, and transforming or transfecting the expression vector into the host cell;
step B): performing protein expression by using a product in step A); and
step C): purifying an expression product obtained in step B) to obtain the recombinant SARS-CoV-2 RBD trimer protein or the fusion protein.

In step A), the nucleic acid molecule includes a nucleotide sequence encoding the recombinant SARS-CoV-2 RBD trimer protein or encoding the fusion protein.

Preferably, in one embodiment of the present invention, the above nucleotide sequences are shown in SEQ ID NO.9 to SEQ ID NO.17 or sequences with more than 95% of homology with the amino acid sequences shown in SEQ ID NO.9 to SEQ ID NO.17.

The nucleic acid molecule may be prepared according to the nucleotide sequences in the specification by arbitrary suitable molecular biology method.

The expression vector constructed in step A) may use arbitrary suitable method to construct the nucleotide sequences in the expression vector corresponding to the host cell.

Then, the expression vector is transformed or transfected into the host cell. Preferably, in one embodiment of the present invention, after CHO cell expression vector is constructed, the inventor transfects the expression vector into an HEK293FT cell or CHO cell to construct a recombinant cell line.

The protein expression in step B) may express the recombinant protein according to the used different expression systems. Further, in one embodiment of the present invention, the inventor performs screening with a limited dilution method to obtain a cell line capable of stably secrete and express the RBD trimer protein or the fusion protein.

The purifying in step C) may be an arbitrary suitable method, for example, a salting out method, a precipitation method, dialysis or ultrafiltration, molecular sieve chromatography, ion exchange chromatography, hydrophobic chromatography, affinity chromatography and the like. Preferably, in one embodiment of the present invention, the RBD trimer protein or the fusion protein is purified using ion exchange chromatography and hydrophobic chromatography.

Certainly, according to the prior art, before the purifying step, the preparation method should further include a collection process of target proteins, for example, collection of a cell culture supernatant rich in the target proteins. The process of breaking the host cell after the target proteins are expressed may use, for example, ultrasonic disruption, breaking with repeated freeze thawing, a chemical treatment method or other arbitrary suitable breaking methods. The collection process of the host cell should also be understood as falling within the scope of the purifying.

Another aspect of the present invention provides use of the recombinant SARS-CoV-2 RBD trimer protein, the fusion protein, the nucleic acid molecule, the vector or the host cell to preparation of a drug for treating and/or preventing SARS-CoV-2 infection and/or diseases caused by SARS-CoV-2.

The disease caused by SARS-CoV-2, is preferably, novel coronavirus pneumonia (COVID-19).

Another aspect of the present invention provides a vaccine. The vaccine includes the recombinant SARS-CoV-2 RBD trimer protein or the fusion protein, and the adjuvant.

In the embodiments of the present invention, the vaccine is a recombinant protein vaccine (or called a genetic engineering subunit vaccine). Further, in some other embodiments of the present invention, the vaccine may further be a genetic engineering vector vaccine or a nucleic acid vaccine. The above vaccine includes the nucleotide sequence in this specification or encodes the amino acid sequence in the specification.

The vaccine of the present invention may include an arbitrary suitable adjuvant. However, preferably, in the embodiments of the present invention, the adjuvant is aluminium hydroxide, aluminium phosphate, MF59 or CpG. More preferably, the adjuvant is the aluminium hydroxide.

Another aspect of the present invention provides a method for preparing the vaccine. The recombinant SARS-CoV-2 RBD trimer protein or the fusion protein obtained through purification and the adjuvant are mixed.

Another aspect of the present invention provides use of the vaccine for treating and/or preventing SARS-CoV-2 infection and/or diseases caused by SARS-CoV-2.

The disease caused by SARS-CoV-2, is preferably, novel coronavirus pneumonia (COVID-19).

Another aspect of the present invention provides a drug composition. The drug composition includes the vaccine, and a pharmaceutically acceptable vector.

The pharmaceutically acceptable vector may be an arbitrary pharmaceutically acceptable additive, for example, normal saline, a cell culture medium, glucose, water for injection, glycerol, amino acid, a combination thereof, a stabilizing agent, a surfactant, a preservative, an isotonic agent and the like.

The drug composition provided by the present invention may further be used in combination with other drugs for treating and/or preventing SARS-CoV-2 infection and/or the disease caused by the SARS-CoV-2 with an effective and safe dosage.

Another aspect of the present invention provides a method for eliciting immune response of a subject against various SARS-CoV-2 epidemic strains or treating SARS-CoV-2 infection of a subject, where the vaccine or the drug composition with an effective dose is applied to the subject.

The subject may be a human or other animals.

Application may be intramuscular injection, intraperitoneal injection or subcutaneous injection.

Beneficial effects of the present invention:
the vaccine prepared by the present invention takes the RBD trimer protein as an antigen and is supplemented with an adjuvant to immunize an organism, so that a high-titer neutralizing antibody aiming at various SARS-CoV-2 epidemic strains can be generated at the same time, and can be used for treating and/or preventing SARS-CoV-2 infection and/or coronavirus disease.

### BRIEF DESCRIPTION OF DRAWINGS

FIG. 1 is structure analysis diagram of an SARS-CoV-2 S protein in Example 1 of the present invention, where A is an S protein monomer structure (drawn using a UCSF Chimera software based on a coordinate file with a PDB code being 6zgg); an S1 structure unit includes NTD, RBD, CTD1 and CTD2 structural domains; and B is a composite structure of the RBD and an ACE2 receptor (drawn using the UCSF Chimera software based on a coordinate file with the PDB code being 6m0j);

FIG. 2 is a homologous modeling result diagram of a trimer protein A according to Example 1 of the present invention;

FIG. 3 is an SDS-PAGE detection result according to Example 2 of the present invention, where a lane 1 is a trimer protein, a lane 2 is a trimer protein A, a lane 3 is a trimer protein B, a lane 4 is a trimer protein C, and M is a protein marker (with a molecular weight standard being kDa: 250, 130, 100, 70, 55, 35, 25, 15 and 10);

FIG. 4 is a Western-blot identification diagram of the trimer protein obtained through purification according to Example 2 of the present invention, where a lane 1 is a trimer protein, a lane 2 is a trimer protein A, a lane 3 is a trimer protein B, a lane 4 is a trimer protein C, and M is a protein marker (with a molecular weight standard being kDa: 250, 130, 100, 70, 55, 35, 25, 15 and 10);

FIG. 5 is a binding curve diagram of a recombinant expressed protein in Example 3 of the present invention and an MM43 neutralizing monoclonal antibody;

FIG. 6 is a binding curve diagram of a recombinant expressed protein in Example 3 of the present invention and an MM57 neutralizing monoclonal antibody;

FIG. 7 is a binding curve diagram of a recombinant expressed protein in Example 3 of the present invention and an R001 neutralizing monoclonal antibody;

FIG. 8 is a binding curve diagram of a recombinant expressed protein in Example 3 of the present invention and an R117 neutralizing monoclonal antibody;

FIG. 9 is an SDS-PAGE detection result diagram of a trimer protein A in Example 4 of the present invention, where a lane 1 to a lane 4 are trimer proteins with different protein concentrations, a lane 5 to a lane 8 are trimer proteins A with different protein concentrations, and M is a protein marker (with a molecular weight standard being kDa: 250, 130, 100, 70, 55, 35, 25, 15 and 10);

FIG. 10 is a Western-blot detection result diagram of a trimer protein A in Example 4 of the present invention, where a lane 1 to a lane 4 are trimer proteins with different protein concentrations, a lane 5 to a lane 8 are trimer proteins A with different protein concentrations, and M is a protein marker (with a molecular weight standard being kDa: 250, 130, 100, 70, 55, 35, 25, 15 and 10);

FIG. 11 is a heat stability result diagram of detecting a trimer protein A using a calorimetric differential scanning technology according to Example 4 of the present invention:

FIG. 12 is a result diagram of observing the form of a trimer protein A by a transmission electron microscope according to Example 4 of the present invention:

FIG. 13 is a result diagram of detecting the affinity of a trimer protein A and an hACE2 receptor using surface plasmon resonance technology according to Example 4 of the present invention; and

FIG. 14 is a result diagram of detecting the neutralizing antibody titer of mouse immune serum using a pseudovirus trace neutralization test according to Example 6 of the present invention.

### SEQUENCE DESCRIPTION

SEQ ID NO.1 is an amino acid sequence of one SARS-CoV-2 (prototype strain) RBD in the examples of the present invention:

SEQ ID NO.2 is an amino acid sequence of another SARS-CoV-2 (Beta (B.1.351) mutant strain) RBD in the examples of the present invention;

SEQ ID NO.3 is an amino acid sequence of another SARS-CoV-2 (Kappa(B.1.617.1) mutant strain) RBD in the examples of the present invention;

SEQ ID NO.4 is an amino acid sequence of a trimer protein A in the examples of the present invention;

SEQ ID NO.5 is an amino acid sequence of a trimer protein B in the examples of the present invention;

SEQ ID NO.6 is an amino acid sequence of a trimer protein C in the examples of the present invention;

SEQ ID NO.7 is an amino acid sequence of a trimer protein D in the examples of the present invention;

SEQ ID NO.8 is an amino acid sequence of a trimer protein E in the examples of the present invention;

SEQ ID NO.9 is an optimized nucleotide sequence encoding a trimer protein A in the examples of the present invention;

SEQ ID NO.10 is an optimized nucleotide sequence encoding a trimer protein B in the examples of the present invention;

SEQ ID NO.11 is an optimized nucleotide sequence encoding a trimer protein C in the examples of the present invention;

SEQ ID NO.12 is an optimized nucleotide sequence encoding a trimer protein C in the examples of the present invention;

SEQ ID NO.13 is an optimized nucleotide sequence encoding a trimer protein C in the examples of the present invention;

SEQ ID NO.14 is an optimized nucleotide sequence encoding a trimer protein C in the examples of the present invention;

SEQ ID NO.15 is an optimized nucleotide sequence encoding a trimer protein D in the examples of the present invention;

SEQ ID NO.16 is an optimized nucleotide sequence encoding a trimer protein E in the examples of the present invention;

SEQ ID NO.17 is an optimized nucleotide sequence encoding a trimer protein E in the examples of the present invention;

SEQ ID NO.18 is an amino acid sequence of a trimer protein in the examples of the present invention; and

SEQ ID NO.19 is an amino acid sequence of a dimer protein in the examples of the present invention.

SEQUENCE TABLE

| Number | Sequence |
|---|---|
| SEQ ID NO. 1 | RVQPTESIVRFPNITNLCPFGEVFNATRFA |
| | SVYAWNRKRISNCVADYSVLYNSASFSTFK |
| | CYGVSPTKLNDLCFTNVYADSFVIRGDEVR |
| | QIAPGQTGKIADYNYKLPDDFTGCVIAWNS |
| | NNLDSKVGGNYNYLYRLFRKSNLKPFERDI |
| | STEIYQAGSTPCNGVEGFNCYFPLQSYGFQ |
| | PTNGVGYQPYRVVVLSFELLHAPATVCGPK |
| | KSTNLVKNK |
| SEQ ID NO. 2 | RVQPTESIVRFPNITNLCPFGEVENATRFA |
| | SVYAWNRKRISNCVADYSVLYNSASFSTFK |
| | CYGVSPTKLNDLCFTNVYADSFVIRGDEVR |
| | QIAPGQTGNIADYNYKLPDDFTGCVIAWNS |
| | NNLDSKVGGNYNYLYRLFRKSNLKPFERDI |
| | STEIYQAGSTPCNGVKGFNCYFPLQSYGFQ |
| | PTYGVGYQPYRVVVLSFELLHAPATVCGPK |
| | KSTNLVKNK |
| SEQ ID No. 3 | RVQPTESIVRFPNITNLCPFGEVFNATRFA |
| | SVYAWNRKRISNCVADYSVLYNSASFSTFK |
| | CYGVSPTKLNDLCFTNVYADSFVIRGDEVR |
| | QIAPGQTGKIADYNYKLPDDFTGCVIAWNS |
| | NNLDSKVGGNYNYRYRLFRKSNLKPFERDI |
| | STEIYQAGSTPCNGVQGFNCYFPLQSYGFQ |
| | PTNGVGYQPYRVVVLSFELLHAPATVCGPK |
| | KSTNLVKNK |
| SEQ ID No. 4 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGKIADYNYKLPDDFTGCVIAWN |
| | SNNLDSKVGGNYNYLYRLFRKSNLKPFERD |
| | ISTEIYQAGSTPCNGVEGFNCYFPLQSYGF |
| | QPTNGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGNIADYNYKLPDD |
| | FTGCVIAWNSNNLDSKVGGNYNYLYRLFRK |
| | SNLKPFERDISTEIYQAGSTPCNGVKGFNC |
| | YFPLQSYGFQPTYGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGKIA |
| | DYNYKLPDDFTGCVIAWNSNNLDSKVGGNY |
| | NYRYRLFRKSNLKPFERDISTEIYQAGSTP |
| | CNGVQGFNCYFPLQSYGFQPTNGVGYQPYR |
| | VVVLSFELLHAPATVCGPKKSTNLVKNK |
| SEQ ID No. 5 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGKIADYNYKLPDDFTGCVIAWN |
| | SNNLDSKVGGNYNYLYRLFRKSNLKPFERD |
| | ISTEIYQAGSTPCNGVEGFNCYFPLQSYGF |
| | QPTNGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGNIADYNYKLPDD |
| | FTGCVIAWNSNNLDSKVGGNYNYLYRLFRK |
| | SNLKPFERDISTEIYQAGSTPCNGVKGFNC |
| | YFPLQSYGFQPTYGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGKIA |
| | DYNYKLPDDFTGCVIAWNSNNLDSKVGGNY |
| | NYLYRLFRKSNLKPFERDISTEIYQAGSTP |
| | CNGVEGENCYFPLQSYGFQPTNGVGYQPYR |
| | VVVLSFELLHAPATVCGPKKSTNLVKNK |
| SEQ ID No. 6 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGKIADYNYKLPDDFTGCVIAWN |
| | SNNLDSKVGGNYNYLYRLFRKSNLKPFERD |
| | ISTEIYQAGSTPCNGVEGFNCYFPLQSYGF |
| | QPTNGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGNIADYNYKLPDD |
| | FTGCVIAWNSNNLDSKVGGNYNYLYRLFRK |
| | SNLKPFERDISTEIYQAGSTPCNGVKGFNC |
| | YFPLQSYGFQPTYGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGNIA |
| | DYNYKLPDDFTGCVIAWNSNNLDSKVGGNY |
| | NYLYRLFRKSNLKPFERDISTEIYQAGSTP |
| | CNGVKGFNCYFPLQSYGFQPTYGVGYQPYR |
| | VVVLSFELLHAPATVCGPKKSTNLVKNK |
| SEQ ID No. 7 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGNIADYNYKLPDDFTGCVIAWN |
| | SNNLDSKVGGNYNYLYRLFRKSNLKPFERD |
| | ISTEIYQAGSTPCNGVKGFNCYFPLQSYGF |
| | QPTYGVGYQPYRVVVLSFELLHAPATVCGP |
| | KKSTNLVKNKRVQPTESIVRFPNITNLCPF |
| | GEVENATRFASVYAWNRKRISNCVADYSVL |
| | YNSASFSTFKCYGVSPTKLNDLCFTNVYAD |
| | SFVIRGDEVRQIAPGQTGKIADYNYKLPDD |
| | FTGCVIAWNSNNLDSKVGGNYNYLYRLFRK |
| | SNLKPFERDISTEIYQAGSTPCNGVEGENC |
| | YFPLQSYGFQPTNGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGNIA |
| | DYNYKLPDDFTGCVIAWNSNNLDSKVGGNY |
| | NYLYRLFRKSNLKPFERDISTEIYQAGSTP |
| | CNGVKGFNCYFPLQSYGFQPTYGVGYQPYR |
| | VVVLSFELLHAPATVCGPKKSTNLVKNK |
| SEQ ID No. 8 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGNIADYNYKLPDDFTGCVIAWN |
| | SNNLDSKVGGNYNYLYRLFRKSNLKPFERD |
| | ISTEIYQAGSTPCNGVKGFNCYFPLQSYGF |
| | QPTYGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGNIADYNYKLPDD |
| | FTGCVIAWNSNNLDSKVGGNYNYLYRLFRK |
| | SNLKPFERDISTEIYQAGSTPCNGVKGFNC |
| | YFPLQSYGFQPTYGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGNIA |
| | DYNYKLPDDFTGCVIAWNSNNLDSKVGGNY |
| | NYLYRLFRKSNLKPFERDISTEIYQAGSTP |
| | CNGVKGFNCYFPLQSYGFQPTYGVGYQPYR |
| | VVVLSFELLHAPATVCGPKKSTNLVKNK |
| SEQ ID No. 9 | ATGAGAGTGCAGCCTACCGAGAGCATTGTC |
| | AGATTCCCTAACATCACCAATCTGTGCCCT |
| | TTCGGCGAGGTGTTCAACGCTACAAGATTT |
| | GCTAGCGTGTACGCTTGGAACAGAAAGAGA |
| | ATCAGCAATTGCGTCGCCGACTACAGCGTG |
| | CTGTACAACAGCGCCTCCTTCAGCACCTTC |
| | AAGTGCTACGGCGTCAGCCCTACAAAACTG |
| | AATGACCTGTGCTTTACAAATGTGTATGCC |
| | GATAGCTTCGTCATCAGAGGCGACGAGGTG |
| | AGGCAAATCGCTCCTGGCCAAACCGGCAAG |
| | ATCGCCGACTACAATTACAAGCTGCCTGAC |
| | GATTTCACCGGATGCGTGATTGCCTGGAAC |
| | AGCAACAACCTGGACAGCAAGGTGGGCGGC |
| | AACTACAACTACCTGTACAGACTGTTTAGA |
| | AAAAGCAACCTGAAGCCTTTCGAGAGAGAC |
| | ATCAGCACCGAGATCTACCAAGCCGGCAGC |
| | ACACCTTGCAACGGCGTGGAGGGATTCAAC |
| | TGCTATTTCCCTCTGCAGAGCTACGGCTTT |
| | CAGCCTACCAACGGCGTGGGCTATCAGCCT |
| | TATAGAGTCGTGGTGCTCAGCTTCGAACTC |
| | CTGCACGCCCCTGCTACAGTGTGTGGCCCT |
| | AAGAAGTCCACCAACCTGGTGAAGAATAAG |
| | AGAGTGCAACCTACCGAGAGCATCGTGAGA |
| | TTCCCCAATATTACCAACCTGTGCCCCTTT |
| | GGCGAAGTGTTCAACGCCACAAGATTCGCT |
| | AGCGTGTACGCCTGGAACAGAAAGAGAATC |
| | AGCAACTGCGTGGCCGACTACAGCGTGCTC |
| | TACAACTCCGCTAGCTTCAGCACCTTTAAG |
| | TGCTACGGCGTGAGCCCTACCAAGCTGAAC |
| | GACCTGTGCTTTACCAACGTCTATGCTGAC |
| | AGCTTTGTGATCAGAGGCGACGAGGTGAGA |
| | CAGATCGCTCCCGGACAGACCGGCAATATC |
| | GCCGATTACAACTACAAGCTGCCCGACGAT |
| | TTCACCGGATGCGTGATCGCCTGGAACAGC |
| | AACAATCTGGACTCCAAGGTGGGAGGCAAT |
| | TACAATTACCTGTACAGACTGTTTAGAAAA |
| | TCCAACCTGAAGCCTTTCGAGAGAGACATT |
| | TCCACCGAGATCTACCAAGCCGGCTCCACA |
| | CCTTGCAATGGAGTGAAGGGCTTCAACTGC |
| | TACTTCCCTCTGCAGAGCTACGGCTTTCAG |
| | CCTACATACGGAGTCGGCTATCAGCCTTAC |
| | AGAGTCGTCGTCCTGAGCTTCGAGCTCCTG |
| | CACGCCCCCGCCACCGTCTGCGGACCTAAG |
| | AAGTCCACAAACCTCGTCAAAAACAAGAGA |
| | GTGCAGCCTACCGAGAGCATCGTGAGGTTT |
| | CCTAACATCACCAACCTGTGCCCTTTCGGC |
| | GAGGTCTTTAACGCCACAAGATTCGCTAGC |
| | GTCTACGCCTGGAACAGAAAGAGGATTAGC |
| | AATTGTGTCGCCGACTACAGCGTGCTGTAC |
| | AACAGCGCTAGCTTCAGCACCTTCAAGTGC |
| | TACGGCGTGAGCCCTACCAAGCTGAACGAC |
| | CTGTGCTTTACAAACGTGTATGCCGACAGC |
| | TTCGTGATTAGGGGCGACGAGGTGAGGCAA |
| | ATCGCTCCTGGCCAAACCGGCAAGATCGCC |
| | GACTACAACTACAAGCTGCCTGACGATTTC |
| | ACCGGCTGCGTGATCGCCTGGAACAGCAAC |
| | AACCTGGATAGCAAGGTGGGCGGCAATTAC |
| | AACTACAGATATAGACTGTTCAGAAAGAGC |
| | AACCTGAAGCCTTTCGAGAGAGACATCTCC |
| | ACCGAGATCTATCAAGCCGGCAGCACACCT |
| | TGCAACGGAGTGCAAGGCTTCAACTGCTAT |
| | TTCCCTCTGCAATCCTACGGCTTTCAGCCT |
| | ACCAACGGAGTGGGCTATCAGCCTTACAGA |
| | GTCGTCGTGCTGAGCTTCGAGCTGCTGCAC |
| | GCCCCTGCCACAGTGTGCGGACCTAAAAAG |
| | AGCACAAATCTGGTGAAGAACAAGTGA |
| SEQ ID No. 10 | ATGAGAGTGCAGCCTACAGAGAGCATTGTG |
| | AGATTCCCTAACATCACAAACCTGTGCCCT |
| | TTCGGCGAGGTGTTCAACGCCACAAGATTC |
| | GCTAGCGTGTACGCCTGGAACAGAAAAAGA |
| | ATTAGCAATTGCGTCGCCGATTACAGCGTG |
| | CTGTATAACAGCGCTTCCTTCAGCACCTTC |
| | AAGTGCTACGGCGTCAGCCCTACAAAGCTG |
| | AACGACCTGTGCTTCACAAACGTGTATGCC |
| | GACAGCTTCGTGATCAGAGGCGACGAGGTG |
| | AGGCAAATCGCTCCTGGCCAAACCGGCAAG |
| | ATCGCCGACTACAATTACAAGCTGCCTGAT |
| | GACTTCACCGGATGCGTGATTGCCTGGAAC |
| | AGCAACAACCTGGACAGCAAAGTCGGAGGA |
| | AACTACAACTACCTGTACAGACTGTTCAGA |
| | AAGAGCAACCTGAAGCCTTTCGAGAGAGAC |
| | ATCAGCACCGAGATCTACCAAGCCGGCTCC |
| | ACACCTTGCAACGGCGTGGAGGGATTCAAC |
| | TGCTACTTCCCTCTGCAGAGCTACGGCTTT |
| | CAGCCCACCAACGGCGTGGGCTATCAGCCT |
| | TACAGAGTGGTCGTCCTGAGCTTTGAGCTG |
| | CTCCACGCCCCTGCCACCGTCTGCGGCCCC |
| | AAAAAAAGCACCAATCTGGTGAAGAACAAG |
| | AGGGTGCAGCCTACAGAGAGCATCGTGAGA |
| | TTCCCTAACATTACCAACCTGTGCCCTTTC |
| | GGAGAAGTGTTTAACGCCACAAGATTCGCC |
| | TCCGTCTACGCTTGGAATAGAAAGAGGATC |
| | AGCAACTGCGTGGCCGACTACAGCGTGCTG |
| | TACAATTCCGCCTCCTTCAGCACCTTCAAG |
| | TGTTACGGCGTGAGCCCTACCAAGCTGAAC |
| | GACCTGTGCTTCACCAACGTGTACGCCGAT |
| | AGCTTCGTGATTAGAGGCGACGAGGTGAGG |
| | CAGATCGCCCCTGGACAAACCGGCAACATT |
| | GCTGACTACAATTACAAGCTGCCTGACGAC |
| | TTCACCGGCTGCGTGATCGCCTGGAACAGC |
| | AACAACCTGGACAGCAAGGTCGGAGGCAAT |
| | TACAATTACCTCTATAGACTGTTCAGAAAA |
| | AGCAATCTCAAGCCTTTCGAAAGAGACATT |
| | AGCACCGAGATCTACCAAGCCGGCAGCACC |
| | CCCTGCAACGGCGTGAAAGGATTCAACTGC |
| | TACTTCCCCCTGCAGAGCTACGGCTTTCAG |
| | CCCACCTACGGAGTGGGCTATCAACCCTAC |
| | AGAGTGGTCGTGCTCTCCTTCGAGCTGCTG |
| | CATGCCCCTGCCACAGTGTGCGGACCTAAA |
| | AAGTCCACCAACCTCGTGAAGAACAAGAGA |
| | GTGCAGCCTACAGAGAGCATCGTGAGGTTC |
| | CCCAACATCACCAACCTGTGCCCTTTCGGC |
| | GAGGTGTTTAACGCTACAAGATTCGCTAGC |
| | GTGTACGCTTGGAACAGAAAGAGAATCTCC |
| | AATTGCGTGGCCGACTACAGCGTGCTGTAC |
| | AACAGCGCTAGCTTCAGCACCTTCAAGTGC |
| | TACGGAGTGTCCCCTACCAAGCTGAACGAC |
| | CTGTGCTTCACCAACGTGTACGCCGATAGC |
| | TTCGTGATTAGAGGAGACGAAGTGAGACAG |
| | ATTGCTCCTGGACAGACCGGCAAGATCGCC |
| | GACTACAATTACAAGCTGCCTGATGACTTT |
| | ACCGGCTGTGTGATTGCCTGGAACAGCAAC |
| | AACCTGGACAGCAAGGTGGGCGGCAACTAC |
| | AACTATCTGTACAGACTCTTCAGAAAGAGC |
| | AATCTGAAGCCTTTTGAAAGGGACATCAGC |
| | ACCGAGATCTATCAAGCCGGCAGCACCCCT |
| | TGCAACGGAGTCGAGGGCTTCAACTGCTAC |
| | TTTCCTCTGCAGAGCTATGGCTTTCAGCCT |
| | ACCAACGGCGTCGGATATCAGCCTTACAGA |
| | GTCGTGGTGCTGAGCTTCGAGCTGCTCCAC |
| | GCCCCTGCCACCGTCTGCGGCCCTAAGAAA |
| | AGCACCAACCTGGTCAAGAACAAATGA |
| SEQ ID No. 11 | ATGAGAGTGCAGCCTACAGAGAGCATTGTG |
| | AGATTCCCTAACATCACCAATCTGTGCCCT |
| | TTCGGCGAGGTGTTCAACGCCACAAGATTC |
| | GCTAGCGTGTACGCCTGGAACAGAAAGAGG |
| | ATCTCCAATTGCGTGGCTGACTATAGCGTG |
| | CTGTACAATAGCGCTAGCTTCAGCACCTTC |
| | AAGTGCTACGGCGTCAGCCCTACAAAGCTG |
| | AACGACCTGTGCTTCACAAACGTGTATGCC |
| | GACAGCTTCGTGATCAGAGGCGACGAGGTG |
| | AGGCAAATCGCTCCTGGCCAAACCGGCAAG |
| | ATCGCCGACTATAATTATAAGCTGCCTGAT |
| | GACTTCACCGGCTGCGTCATCGCCTGGAAC |
| | AGCAATAATCTGGACAGCAAGGTCGGAGGC |
| | AACTACAACTACCTGTACAGACTGTTCAGA |
| | AAGAGCAACCTGAAGCCTTTCGAGAGAGAC |
| | ATCAGCACCGAGATTTACCAAGCCGGCTCC |
| | ACCCCTTGCAACGGCGTGGAAGGCTTCAAC |
| | TGCTACTTCCCTCTGCAGTCCTACGGCTTT |
| | CAGCCTACAAACGGCGTGGGCTACCAACCC |
| | TACAGAGTCGTGGTGCTCAGCTTCGAGCTG |
| | CTGCATGCCCCTGCTACCGTGTGCGGCCCC |
| | AAAAAGAGCACCAACCTCGTGAAGAATAAG |
| | AGAGTGCAGCCTACCGAGAGCATCGTCAGA |
| | TTCCCTAACATCACCAATCTCTGCCCCTTC |
| | GGCGAGGTGTTCAACGCCACAAGATTCGCT |
| | AGCGTCTACGCTTGGAATAGAAAGAGAATC |
| | AGCAACTGTGTGGCCGACTATAGCGTGCTG |
| | TACAACAGCGCTAGCTTTAGCACCTTTAAG |
| | TGCTACGGCGTGTCCCCTACCAAGCTGAAC |
| | GACCTGTGCTTCACAAATGTGTACGCCGAC |
| | AGCTTCGTGATCAGAGGAGACGAGGTGAGG |
| | CAGATCGCCCCTGGACAAACCGGCAATATT |
| | GCCGACTACAACTACAAGCTGCCCGACGAC |
| | TTCACCGGCTGCGTGATCGCCTGGAACAGC |
| | AACAACCTCGACAGCAAAGTGGGAGGCAAT |
| | TACAACTACCTGTATAGACTGTTTAGAAAG |
| | AGCAACCTGAAGCCTTTCGAGAGGGACATC |
| | TCCACCGAGATCTACCAAGCCGGCAGCACC |
| | CCTTGTAACGGCGTGAAGGGCTTCAACTGT |
| | TACTTCCCTCTGCAGAGCTATGGCTTTCAG |
| | CCTACATACGGAGTGGGCTATCAGCCTTAC |
| | AGAGTGGTGGTCCTCTCCTTTGAACTCCTG |
| | CATGCCCCTGCCACAGTGTGCGGACCTAAA |
| | AAGAGCACCAACCTCGTGAAGAATAAGAGA |
| | GTGCAGCCTACCGAGAGCATCGTGAGATTC |
| | CCCAATATCACCAACCTGTGTCCTTTCGGC |
| | GAGGTGTTCAATGCCACAAGATTCGCTAGC |
| | GTCTATGCCTGGAACAGAAAGAGAATCTCC |
| | AATTGCGTGGCCGACTACAGCGTGCTGTAC |
| | AACAGCGCTAGCTTCAGCACCTTCAAGTGC |
| | TACGGAGTGAGCCCTACCAAGCTGAACGAC |
| | CTCTGCTTTACCAATGTGTACGCCGATAGC |
| | TTCGTCATTAGAGGAGACGAGGTGAGACAG |
| | ATTGCTCCTGGACAGACCGGCAACATCGCC |
| | GACTACAACTACAAGCTGCCTGACGATTTT |
| | ACCGGCTGTGTGATCGCCTGGAACAGCAAC |
| | AACCTGGACAGCAAGGTCGGCGGCAACTAC |
| | AACTATCTGTACAGACTGTTTAGAAAGAGC |
| | AACCTGAAGCCTTTCGAAAGGGACATCAGC |
| | ACCGAGATCTATCAAGCCGGCTCCACCCCT |
| | TGCAACGGCGTCAAGGGCTTTAACTGCTAC |
| | TTCCCTCTGCAGAGCTACGGCTTTCAGCCT |
| | ACCTACGGCGTCGGATATCAGCCTTATAGA |
| | GTGGTCGTGCTGAGCTTCGAGCTGCTCCAC |
| | GCCCCTGCCACCGTCTGCGGCCCTAAAAAG |
| | AGCACCAACCTGGTCAAGAACAAATGA |
| SEQ ID No. 12 | ATGAGAGTGCAGCCCACAGAGAGCATTGTG |
| | AGATTCCCCAACATCACCAATCTGTGCCCC |
| | TTCGGCGAGGTGTTCAACGCCACAAGATTC |
| | GCTAGCGTGTACGCCTGGAACAGAAAGCGG |
| | ATCTCCAATTGCGTGGCTGACTATAGCGTG |
| | CTGTACAATAGCGCTAGCTTCAGCACCTTC |
| | AAGTGCTACGGCGTCAGCCCCACAAAGCTG |
| | AACGACCTGTGCTTCACAAACGTGTATGCC |
| | GACAGCTTCGTGATCAGAGGCGACGAGGTG |
| | CGGCAAATCGCTCCCGGCCAAACCGGCAAG |
| | ATCGCCGACTATAATTATAAGCTGCCCGAT |
| | GACTTCACCGGCTGCGTCATCGCCTGGAAC |
| | AGCAATAATCTGGACAGCAAGGTCGGGGGC |
| | AACTACAACTACCTGTACAGACTGTTCAGA |
| | AAGAGCAACCTGAAGCCCTTCGAGAGAGAC |
| | ATCAGCACCGAGATTTACCAAGCCGGCTCC |
| | ACCCCCTGCAACGGCGTGGAAGGCTTCAAC |
| | TGCTACTTCCCCCTGCAGTCCTACGGCTTT |
| | CAGCCCACAAACGGCGTGGGCTACCAACCT |
| | TACAGAGTCGTGGTGCTCAGCTTCGAGCTG |
| | CTGCATGCCCCCGCTACCGTGTGCGGCCCT |
| | AAAAAGAGCACCAACCTCGTGAAGAATAAG |
| | AGAGTGCAGCCCACCGAGAGCATCGTCAGA |
| | TTCCCCAACATCACCAATCTCTGCCCTTTC |
| | GGCGAGGTGTTCAACGCCACAAGATTCGCT |
| | AGCGTCTACGCTTGGAACCGGAAGAGAATC |
| | AGCAACTGTGTGGCCGACTATAGCGTGCTG |
| | TACAACAGCGCTAGCTTTAGCACCTTTAAG |
| | TGCTACGGCGTGTCCCCCACCAAGCTGAAC |
| | GACCTGTGCTTCACAAATGTGTACGCCGAC |
| | AGCTTCGTGATCAGAGGGGACGAGGTGCGG |
| | CAGATCGCCCCCGGGCAAACCGGCAATATT |
| | GCCGACTACAACTACAAGCTGCCTGACGAC |
| | TTCACCGGCTGCGTGATCGCCTGGAACAGC |
| | AACAACCTCGACAGCAAAGTGGGGGGCAAT |
| | TACAACTACCTGTATAGACTGTTTAGAAAG |
| | AGCAACCTGAAGCCCTTCGAGCGGGACATC |
| | TCCACCGAGATCTACCAAGCCGGCAGCACC |
| | CCCTGTAACGGCGTGAAGGGCTTCAACTGT |
| | TACTTCCCCCTGCAGAGCTATGGCTTTCAG |
| | CCCACATACGGGGTGGGCTATCAGCCCTAC |
| | AGAGTGGTGGTCCTCTCCTTTGAACTCCTG |
| | CATGCCCCCGCCACAGTGTGCGGGCCCAAA |
| | AAGAGCACCAACCTCGTGAAGAATAAGAGA |
| | GTGCAGCCCACCGAGAGCATCGTGAGATTC |
| | CCTAATATCACCAACCTGTGTCCCTTCGGC |
| | GAGGTGTTCAATGCCACAAGATTCGCTAGC |
| | GTCTATGCCTGGAACAGAAAGAGAATCTCC |
| | AATTGCGTGGCCGACTACAGCGTGCTGTAC |
| | AACAGCGCTAGCTTCAGCACCTTCAAGTGC |
| | TACGGGGTGAGCCCCACCAAGCTGAACGAC |
| | CTCTGCTTTACCAATGTGTACGCCGATAGC |
| | TTCGTCATCCGGGGGGACGAGGTGAGACAG |
| | ATTGCTCCCGGGCAGACCGGCAACATCGCC |
| | GACTACAACTACAAGCTGCCCGACGATTTT |
| | ACCGGCTGTGTGATCGCCTGGAACAGCAAC |
| | AACCTGGACAGCAAGGTCGGCGGCAACTAC |
| | AACTATCTGTACAGACTGTTTAGAAAGAGC |
| | AACCTGAAGCCCTTCGAACGGGACATCAGC |
| | ACCGAGATCTATCAAGCCGGCTCCACCCCC |
| | TGCAACGGCGTCAAGGGCTTTAACTGCTAC |
| | TTCCCCCTGCAGAGCTACGGCTTTCAGCCC |
| | ACCTACGGCGTCGGGTATCAGCCCTACCGG |
| | GTGGTCGTGCTGAGCTTCGAGCTGCTCCAC |
| | GCCCCCGCCACCGTCTGCGGCCCCAAAAAG |
| | AGCACCAACCTGGTCAAGAACAAATGA |
| SEQ ID No. 13 | ATGAGAGTGCAGCCTACCGAGAGCATCGTG |
| | AGATTTCCTAATATCACCAATCTCTGCCCC |
| | TTCGGCGAGGTGTTTAACGCCACAAGATTT |
| | GCTAGCGTGTACGCCTGGAACAGAAAAAGA |
| | ATCTCCAACTGCGTGGCCGACTACAGCGTC |
| | CTGTACAATAGCGCTAGCTTCAGCACCTTC |
| | AAGTGCTACGGCGTCTCCCCTACAAAGCTG |
| | AATGACCTGTGTTTTACCAACGTGTATGCC |
| | GACAGCTTCGTGATTAGAGGCGATGAGGTG |
| | AGGCAGATCGCCCCTGGACAGACCGGCAAG |
| | ATCGCTGATTACAACTACAAGCTGCCTGAC |
| | GACTTCACCGGCTGCGTGATCGCCTGGAAC |
| | AGCAACAACCTGGATAGCAAGGTGGGCGGC |
| | AACTACAACTATCTGTACAGACTGTTCAGA |
| | AAAAGCAACCTGAAGCCTTTTGAGAGAGAC |
| | ATCAGCACAGAGATCTACCAAGCCGGCAGC |
| | ACCCCTTGCAACGGCGTGGAAGGATTTAAC |
| | TGCTATTTCCCTCTGCAGAGCTATGGCTTC |
| | CAACCTACCAACGGAGTGGGCTATCAGCCC |
| | TACAGAGTGGTCGTGCTGAGCTTCGAGCTG |
| | CTGCACGCTCCTGCCACCGTGTGCGGCCCC |
| | AAAAAGTCCACCAACCTGGTGAAGAATAAA |
| | AGAGTGCAGCCTACCGAATCCATCGTGAGA |
| | TTCCCTAACATCACCAACCTGTGCCCTTTC |
| | GGCGAGGTCTTCAACGCCACAAGATTTGCT |
| | AGCGTGTACGCCTGGAACAGAAAGAGAATC |
| | AGCAACTGCGTGGCCGACTACAGCGTCCTG |
| | TACAACAGCGCCTCCTTCAGCACCTTCAAA |
| | TGCTACGGCGTGTCCCCTACCAAGCTGAAT |
| | GACCTGTGCTTTACCAACGTGTACGCCGAC |
| | AGCTTTGTGATCAGAGGCGACGAGGTGAGA |
| | CAGATTGCTCCTGGACAGACCGGCAACATT |
| | GCCGATTACAATTACAAGCTGCCTGATGAT |
| | TTCACCGGCTGTGTGATCGCCTGGAACAGC |
| | AACAACCTGGACAGCAAGGTGGGCGGCAAC |
| | TATAATTACCTGTATAGACTGTTCAGAAAA |
| | AGCAACCTGAAGCCTTTCGAGAGAGACATC |
| | TCCACCGAAATTTACCAAGCCGGATCCACA |
| | CCTTGCAACGGCGTGAAGGGCTTCAATTGT |
| | TACTTCCCTCTGCAAAGCTACGGCTTTCAG |
| | CCTACATACGGAGTGGGCTACCAACCCTAC |
| | AGAGTGGTCGTGCTGAGCTTCGAGCTGCTC |
| | CACGCCCCTGCCACCGTGTGCGGCCCTAAA |
| | AAGAGCACCAACCTGGTGAAGAATAAGAGA |
| | GTGCAACCCACAGAGAGCATTGTGAGATTC |
| | CCTAACATCACAAATCTGTGCCCTTTCGGC |
| | GAGGTGTTCAACGCCACAAGATTCGCTAGC |
| | GTGTACGCTTGGAACAGAAAGAGAATCAGC |
| | AACTGTGTGGCTGACTACAGCGTGCTCTAC |
| | AACAGCGCTTCCTTCAGCACCTTTAAATGC |
| | TACGGCGTGAGCCCCACAAAGCTCAACGAC |
| | CTGTGCTTCACCAACGTCTACGCCGACAGC |
| | TTCGTGATTAGAGGAGACGAAGTGAGACAG |
| | ATCGCTCCTGGACAGACCGGCAATATCGCC |
| | GACTACAACTACAAACTGCCTGACGACTTC |
| | ACCGGCTGCGTGATCGCCTGGAACAGCAAT |
| | AATCTCGACAGCAAGGTCGGCGGCAACTAC |
| | AATTACCTGTATAGACTGTTTAGAAAGAGC |
| | AACCTGAAGCCCTTCGAGAGAGACATCTCC |
| | ACCGAGATCTACCAAGCCGGCTCCACACCT |
| | TGCAACGGCGTGAAGGGCTTCAACTGCTAC |
| | TTCCCTCTGCAGAGCTACGGCTTCCAACCT |
| | ACCTACGGAGTGGGCTATCAGCCTTATAGA |
| | GTGGTCGTGCTGAGCTTCGAACTGCTGCAC |
| | GCCCCCGCCACCGTGTGCGGCCCCAAAAAG |
| | AGCACAAACCTGGTGAAGAACAAGTGA |
| SEQ ID No. 14 | ATGAGAGTGCAGCCTACCGAGAGCATCGTG |
| | AGATTCCCTAACATCACAAACCTCTGCCCT |
| | TTTGGAGAGGTCTTCAACGCCACAAGATTC |
| | GCCTCCGTGTATGCCTGGAACAGAAAGAGA |
| | ATCAGCAATTGCGTGGCTGACTACAGCGTG |
| | CTGTACAACTCCGCTAGCTTTAGCACCTTC |
| | AAGTGCTACGGCGTGAGCCCTACCAAACTC |
| | AACGATCTGTGCTTTACCAACGTCTATGCC |
| | GACAGCTTTGTGATCAGAGGCGACGAGGTC |
| | AGACAAATCGCCCCCGGACAGACCGGCAAG |
| | ATCGCCGACTACAACTATAAACTGCCTGAC |
| | GACTTCACCGGCTGCGTGATCGCCTGGAAC |
| | AGCAACAACCTGGACTCCAAGGTGGGCGGC |
| | AACTACAACTACCTGTACAGACTCTTCAGA |
| | AAGAGCAACCTGAAACCTTTCGAAAGAGAC |
| | ATCAGCACCGAAATCTACCAAGCCGGCAGC |
| | ACCCCTTGCAACGGCGTGGAGGGCTTTAAT |
| | TGCTACTTTCCTCTGCAAAGCTACGGCTTT |
| | CAGCCCACCAACGGAGTGGGCTATCAGCCT |
| | TATAGAGTCGTCGTGCTGAGCTTTGAGCTG |
| | CTGCACGCCCCTGCCACCGTCTGCGGCCCT |
| | AAAAAGAGCACAAATCTGGTCAAGAACAAG |
| | AGAGTGCAGCCTACCGAGAGCATTGTGAGA |
| | TTTCCTAACATCACCAATCTGTGCCCTTTC |
| | GGCGAGGTGTTCAATGCCACAAGATTTGCT |
| | AGCGTCTACGCCTGGAACAGAAAAAGAATC |
| | AGCAACTGCGTGGCCGACTACAGCGTGCTG |
| | TACAACTCCGCTAGCTTCAGCACCTTTAAG |
| | TGCTACGGCGTCAGCCCTACCAAACTGAAC |
| | GACCTCTGCTTTACCAACGTGTATGCCGAC |
| | AGCTTCGTGATCAGAGGAGACGAGGTGAGA |
| | CAGATCGCCCCTGGACAGACCGGCAACATC |
| | GCCGATTACAATTACAAACTGCCCGACGAC |
| | TTCACCGGCTGTGTGATTGCTTGGAACTCC |
| | AACAACCTCGACAGCAAGGTGGGCGGCAAC |
| | TACAACTACCTGTATAGACTGTTCAGAAAG |
| | AGCAACCTGAAGCCTTTCGAGAGAGATATC |
| | AGCACCGAGATCTACCAAGCCGGCAGCACA |
| | CCTTGTAACGGCGTGAAAGGCTTCAATTGT |
| | TATTTCCCCCTGCAGAGCTACGGCTTTCAG |
| | CCTACCTATGGCGTGGGATACCAACCTTAC |
| | AGAGTGGTCGTGCTGAGCTTCGAACTGCTG |
| | CACGCCCCTGCCACCGTGTGCGGCCCCAAA |
| | AAGAGCACCAATCTGGTGAAGAACAAAAGA |
| | GTGCAACCTACCGAGAGCATCGTGAGATTC |
| | CCCAATATTACAAACCTGTGCCCCTTTGGC |
| | GAGGTGTTCAACGCCACAAGATTCGCTAGC |
| | GTGTACGCCTGGAACAGAAAGAGAATCAGC |
| | AACTGCGTGGCTGACTACAGCGTGCTGTAC |
| | AACAGCGCCTCCTTCAGCACATTCAAGTGC |
| | TACGGAGTCAGCCCTACCAAGCTGAATGAT |
| | CTGTGTTTCACAAACGTGTACGCCGACAGC |
| | TTCGTGATCAGAGGCGACGAAGTGAGACAG |
| | ATTGCCCCTGGACAGACCGGCAACATCGCC |
| | GACTACAATTACAAGCTGCCTGACGATTTC |
| | ACCGGCTGCGTCATCGCCTGGAACAGCAAC |
| | AACCTGGACTCCAAAGTGGGCGGCAACTAC |
| | AATTACCTGTATAGACTGTTCAGAAAGTCC |
| | AATCTCAAGCCCTTCGAGAGAGACATCAGC |
| | ACCGAGATTTATCAAGCCGGCAGCACCCCT |
| | TGCAATGGAGTGAAAGGCTTCAACTGCTAC |
| | TTCCCTCTGCAGAGCTACGGATTTCAGCCT |
| | ACCTACGGAGTGGGCTATCAGCCTTACAGA |
| | GTGGTCGTGCTGAGCTTTGAGCTGCTGCAC |
| | GCTCCTGCCACCGTGTGCGGCCCTAAAAAG |
| | AGCACCAACCTGGTGAAGAACAAGTGA |
| SEQ ID No. 15 | ATGAGAGTGCAGCCTACAGAGAGCATTGTG |
| | AGATTCCCTAACATCACAAACCTGTGCCCT |
| | TTCGGCGAGGTGTTCAACGCCACAAGATTC |
| | GCTAGCGTGTACGCCTGGAACAGAAAAAGA |
| | ATTAGCAATTGCGTCGCCGATTACAGCGTG |
| | CTGTATAACAGCGCTTCCTTCAGCACCTTC |
| | AAGTGCTACGGCGTCAGCCCTACAAAGCTG |
| | AACGACCTGTGCTTCACAAACGTGTATGCC |
| | GACAGCTTCGTGATCAGAGGCGACGAGGTG |
| | AGGCAAATCGCTCCTGGCCAAACCGGCAAC |
| | ATCGCCGACTACAATTACAAGCTGCCTGAT |
| | GACTTCACCGGATGCGTGATTGCCTGGAAC |
| | AGCAACAACCTGGACAGCAAAGTCGGAGGA |
| | AACTACAACTACCTGTACAGACTGTTCAGA |
| | AAGAGCAACCTGAAGCCTTTCGAGAGAGAC |
| | ATCAGCACCGAGATCTACCAAGCCGGCTCC |
| | ACACCTTGCAACGGCGTGAAGGGATTCAAC |
| | TGCTACTTCCCTCTGCAGAGCTACGGCTTT |
| | CAGCCCACCTACGGCGTGGGCTATCAGCCT |
| | TACAGAGTGGTCGTCCTGAGCTTTGAGCTG |
| | CTCCACGCCCCTGCCACCGTCTGCGGCCCC |
| | AAAAAAAGCACCAATCTGGTGAAGAACAAG |
| | AGGGTGCAGCCTACAGAGAGCATCGTGAGA |
| | TTCCCTAACATTACCAACCTGTGCCCTTTC |
| | GGAGAAGTGTTTAACGCCACAAGATTCGCC |
| | TCCGTCTACGCTTGGAATAGAAAGAGGATC |
| | AGCAACTGCGTGGCCGACTACAGCGTGCTG |
| | TACAATTCCGCCTCCTTCAGCACCTTCAAG |
| | TGTTACGGCGTGAGCCCTACCAAGCTGAAC |
| | GACCTGTGCTTCACCAACGTGTACGCCGAT |
| | AGCTTCGTGATTAGAGGCGACGAGGTGAGG |
| | CAGATCGCCCCTGGACAAACCGGCAAGATT |
| | GCTGACTACAATTACAAGCTGCCTGACGAC |
| | TTCACCGGCTGCGTGATCGCCTGGAACAGC |
| | AACAACCTGGACAGCAAGGTCGGAGGCAAT |
| | TACAATTACCTCTATAGACTGTTCAGAAAA |
| | AGCAATCTCAAGCCTTTCGAAAGAGACATT |
| | AGCACCGAGATCTACCAAGCCGGCAGCACC |
| | CCCTGCAACGGCGTGGAAGGATTCAACTGC |
| | TACTTCCCCCTGCAGAGCTACGGCTTTCAG |
| | CCCACCAACGGAGTGGGCTATCAACCCTAC |
| | AGAGTGGTCGTGCTCTCCTTCGAGCTGCTG |
| | CATGCCCCTGCCACAGTGTGCGGACCTAAA |
| | AAGTCCACCAACCTCGTGAAGAACAAGAGA |
| | GTGCAGCCTACAGAGAGCATCGTGAGGTTC |
| | CCCAACATCACCAACCTGTGCCCTTTCGGC |
| | GAGGTGTTTAACGCTACAAGATTCGCTAGC |
| | GTGTACGCTTGGAACAGAAAGAGAATCTCC |
| | AATTGCGTGGCCGACTACAGCGTGCTGTAC |
| | AACAGCGCTAGCTTCAGCACCTTCAAGTGC |
| | TACGGAGTGTCCCCTACCAAGCTGAACGAC |
| | CTGTGCTTCACCAACGTGTACGCCGATAGC |
| | TTCGTGATTAGAGGAGACGAAGTGAGACAG |
| | ATTGCTCCTGGACAGACCGGCAACATCGCC |
| | GACTACAATTACAAGCTGCCTGATGACTTT |
| | ACCGGCTGTGTGATTGCCTGGAACAGCAAC |
| | AACCTGGACAGCAAGGTGGGCGGCAACTAC |
| | AACTATCTGTACAGACTCTTCAGAAAGAGC |
| | AATCTGAAGCCTTTTGAAAGGGACATCAGC |
| | ACCGAGATCTATCAAGCCGGCAGCACCCCT |
| | TGCAACGGAGTCAAGGGCTTCAACTGCTAC |
| | TTTCCTCTGCAGAGCTATGGCTTTCAGCCT |
| | ACCTACGGCGTCGGATATCAGCCTTACAGA |
| | GTCGTGGTGCTGAGCTTCGAGCTGCTCCAC |
| | GCCCCTGCCACCGTCTGCGGCCCTAAGAAA |
| | AGCACCAACCTGGTCAAGAACAAATGA |
| SEQ ID No. 16 | ATGAGGGTGCAGCCCACAGAGAGCATCGTC |
| | AGATTCCCTAACATCACAAACCTGTGTCCT |
| | TTTGGCGAGGTCTTCAACGCCACAAGATTC |
| | GCTAGCGTGTACGCCTGGAATAGAAAAAGG |
| | ATTAGCAACTGTGTGGCTGACTACAGCGTC |
| | CTGTATAACAGCGCTTCCTTTTCCACCTTT |
| | AAGTGTTATGGCGTGTCCCCCACAAAGCTG |
| | AACGACCTGTGTTTTACCAATGTGTACGCC |
| | GACAGCTTCGTCATCAGAGGAGACGAGGTG |
| | AGACAAATCGCCCCTGGACAGACCGGCAAC |
| | ATCGCCGACTACAATTATAAGCTGCCTGAC |
| | GACTTTACCGGCTGCGTGATTGCTTGGAAC |
| | TCCAACAACCTGGATAGCAAGGTGGGAGGA |
| | AACTACAACTACCTCTACAGACTCTTCAGA |
| | AAGAGCAATCTGAAGCCTTTCGAGAGAGAT |
| | ATCTCCACAGAGATCTATCAAGCCGGCAGC |
| | ACCCCTTGTAACGGAGTGAAGGGATTCAAT |
| | TGTTATTTCCCTCTGCAAAGCTATGGATTT |
| | CAGCCTACCTACGGAGTGGGATATCAGCCC |
| | TATAGAGTGGTCGTGCTGAGCTTCGAGCTC |
| | CTGCACGCCCCTGCTACAGTCTGTGGCCCT |
| | AAGAAGTCCACAAACCTCGTGAAGAACAAG |
| | AGAGTGCAGCCTACCGAGAGCATCGTGAGG |
| | TTCCCTAACATTACCAACCTCTGTCCTTTT |
| | GGAGAAGTCTTTAATGCCACAAGATTTGCT |
| | AGCGTGTACGCTTGGAATAGAAAGAGGATT |
| | TCCAATTGCGTGGCCGACTACTCCGTCCTG |
| | TACAACAGCGCTAGCTTCAGCACCTTCAAA |
| | TGCTACGGAGTGAGCCCTACCAAGCTCAAC |
| | GACCTGTGCTTTACCAACGTGTACGCTGAC |
| | AGCTTCGTCATTAGGGGAGACGAGGTGAGG |
| | CAGATCGCTCCTGGACAGACCGGCAACATC |
| | GCCGACTACAACTACAAGCTGCCCGACGAT |
| | TTCACCGGCTGCGTCATTGCCTGGAATAGC |
| | AACAATCTGGACAGCAAGGTGGGAGGAAAC |
| | TATAATTACCTGTACAGACTGTTTAGGAAA |
| | AGCAACCTCAAACCCTTCGAAAGAGACATT |
| | AGCACAGAGATCTACCAAGCCGGCTCCACC |
| | CCCTGCAATGGCGTGAAGGGATTTAATTGT |
| | TACTTCCCCCTGCAGAGCTATGGCTTTCAA |
| | CCTACCTACGGCGTGGGCTATCAGCCCTAT |
| | AGGGTGGTGGTCCTCAGCTTTGAACTCCTG |
| | CACGCTCCTGCCACCGTGTGCGGCCCCAAA |
| | AAGAGCACCAATCTGGTCAAGAACAAGAGA |
| | GTGCAGCCCACAGAGTCCATCGTGAGATTT |
| | CCTAATATTACCAACCTGTGCCCTTTCGGA |
| | GAGGTGTTTAATGCTACAAGATTTGCTAGC |
| | GTCTATGCCTGGAACAGAAAGAGAATCAGC |
| | AACTGCGTGGCCGATTACAGCGTCCTGTAC |
| | AATAGCGCTTCCTTCTCCACCTTTAAATGC |
| | TACGGCGTGAGCCCCACCAAGCTGAATGAC |
| | CTCTGTTTCACCAACGTGTATGCCGACTCC |
| | TTTGTGATTAGAGGAGATGAGGTGAGACAG |
| | ATCGCCCCTGGACAAACCGGCAACATTGCC |
| | GATTACAATTACAAGCTGCCTGATGACTTC |
| | ACCGGCTGTGTGATTGCCTGGAACAGCAAT |
| | AACCTGGACAGCAAGGTGGGCGGCAATTAT |
| | AACTACCTGTATAGACTGTTCAGAAAGAGC |
| | AACCTGAAGCCTTTTGAGAGAGACATCAGC |
| | ACCGAGATTTACCAAGCCGGCTCCACCCCT |
| | TGCAACGGCGTGAAGGGCTTCAATTGCTAC |
| | TTCCCTCTGCAGTCCTACGGCTTTCAGCCT |
| | ACATACGGCGTGGGATATCAGCCTTATAGA |
| | GTCGTGGTGCTCAGCTTCGAGCTGCTGCAC |
| | GCCCCCGCTACAGTGTGTGGACCTAAAAAG |
| | TCCACCAATCTCGTCAAGAATAAGTGA |
| SEQ ID No. 17 | ATGAGAGTGCAGCCTACCGAGAGCATCGTG |
| | AGATTCCCTAACATCACCAACCTCTGCCCT |
| | TTCGGAGAGGTCTTCAACGCCACAAGATTT |
| | GCTAGCGTGTACGCCTGGAACAGAAAAAGA |
| | ATCTCCAACTGCGTGGCCGACTACAGCGTC |
| | CTGTACAATAGCGCTAGCTTCAGCACCTTC |
| | AAGTGCTACGGCGTCTCCCCTACAAAGCTG |
| | AATGACCTGTGCTTCACCAACGTGTATGCC |
| | GACAGCTTCGTGATTAGAGGCGATGAGGTG |
| | AGGCAGATCGCCCCTGGACAAACCGGCAAC |
| | ATCGCCGATTATAACTACAAGCTGCCTGAC |
| | GACTTCACCGGCTGCGTGATCGCCTGGAAC |
| | AGCAACAACCTGGACAGCAAGGTCGGCGGC |
| | AACTACAACTACCTCTACAGACTGTTCAGA |
| | AAGTCCAACCTGAAGCCTTTCGAGAGGGAC |
| | ATCAGCACCGAGATTTACCAAGCCGGCAGC |
| | ACCCCTTGCAACGGCGTGAAGGGATTTAAC |
| | TGCTATTTCCCTCTGCAGAGCTACGGCTTC |
| | CAACCTACCTATGGAGTGGGCTACCAACCC |
| | TACAGAGTGGTGGTCCTGAGCTTCGAACTG |
| | CTGCACGCCCCTGCCACCGTGTGTGGCCCT |
| | AAAAAGAGCACCAATCTCGTGAAAAACAAG |
| | AGAGTGCAGCCTACAGAAAGCATTGTGAGA |
| | TTTCCTAATATCACCAACCTGTGCCCTTTC |
| | GGCGAGGTGTTCAACGCTACAAGATTCGCT |
| | AGCGTGTACGCCTGGAATAGAAAGAGAATC |
| | AGCAACTGCGTGGCCGACTACAGCGTGCTG |
| | TACAACAGCGCTTCCTTTAGCACCTTCAAG |
| | TGTTACGGCGTGAGCCCCACCAAGCTCAAC |
| | GACCTGTGCTTCACAAACGTGTACGCTGAC |
| | AGCTTCGTGATCAGAGGCGATGAGGTGAGA |
| | CAGATCGCCCCTGGCCAAACCGGCAACATC |
| | GCCGATTACAACTATAAGCTCCCTGATGAC |
| | TTCACCGGCTGCGTGATCGCTTGGAACAGC |
| | AACAACCTGGACAGCAAGGTGGGCGGCAAC |
| | TACAACTACCTCTATAGACTGTTCAGAAAA |
| | TCCAACCTGAAGCCTTTCGAGAGAGACATC |
| | TCCACCGAGATTTATCAAGCCGGAAGCACC |
| | CCCTGCAATGGCGTGAAGGGCTTCAACTGC |
| | TACTTCCCTCTCCAATCCTACGGCTTCCAA |
| | CCTACCTACGGAGTGGGCTATCAGCCCTAC |
| | AGAGTGGTCGTGCTGAGCTTCGAGCTCCTG |
| | CACGCCCCTGCCACCGTGTGCGGCCCTAAA |
| | AAGAGCACCAACCTGGTCAAAAATAAGAGA |
| | GTGCAGCCCACCGAGAGCATCGTGAGATTC |
| | CCTAATATCACCAACCTGTGCCCTTTTGGA |
| | GAGGTGTTCAACGCCACAAGATTCGCTAGC |
| | GTGTACGCCTGGAATAGAAAGAGGATCAGC |
| | AACTGCGTGGCCGACTACAGCGTGCTCTAC |
| | AACAGCGCCTCCTTCAGCACCTTTAAGTGT |
| | TACGGCGTCTCCCCTACCAAGCTGAACGAT |
| | CTCTGCTTTACCAACGTGTACGCCGACAGC |
| | TTTGTGATCAGAGGCGACGAAGTGAGGCAA |
| | ATCGCTCCTGGACAGACCGGAAACATCGCC |
| | GACTACAACTACAAGCTGCCTGACGACTTC |
| | ACCGGCTGCGTCATCGCCTGGAATAGCAAC |
| | AACCTGGATAGCAAAGTCGGAGGCAACTAC |
| | AACTACCTGTACAGACTCTTCAGAAAGAGC |
| | AACCTCAAACCTTTCGAGAGAGACATCAGC |
| | ACAGAGATCTACCAAGCCGGCAGCACCCCT |
| | TGCAACGGAGTCAAGGGCTTCAACTGCTAC |
| | TTTCCTCTGCAGAGCTACGGCTTTCAGCCT |
| | ACCTACGGCGTCGGCTATCAGCCTTACAGA |
| | GTGGTCGTGCTCAGCTTCGAACTCCTGCAC |
| | GCCCCCGCCACCGTGTGCGGACCTAAAAAG |
| | AGCACCAATCTGGTGAAGAACAAATGA |
| SEQ ID No. 18 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGKIADYNYKLPDDFTGCVIAWN |
| | SNNLDSKVGGNYNYLYRLFRKSNLKPFERD |
| | ISTEIYQAGSTPCNGVEGFNCYFPLQSYGF |
| | QPTNGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGKIADYNYKLPDD |
| | FTGCVIAWNSNNLDSKVGGNYNYLYRLFRK |
| | SNLKPFERDISTEIYQAGSTPCNGVEGENC |
| | YFPLQSYGFQPTNGVGYQPYRVVVLSFELL |
| | HAPATVCGPKKSTNLVKNKRVQPTESIVRF |
| | PNITNLCPFGEVFNATRFASVYAWNRKRIS |
| | NCVADYSVLYNSASFSTFKCYGVSPTKLND |
| | LCFTNVYADSFVIRGDEVRQIAPGQTGKIA |
| | DYNYKLPDDFTGCVIAWNSNNLDSKVGGNY |
| | NYLYRLFRKSNLKPFERDISTEIYQAGSTP |
| | CNGVEGENCYFPLQSYGFQPTNGVGYQPYR |
| | VVVLSFELLHAPATVCGPKKSTNLVKNK |
| SEQ ID No. 19 | MRVQPTESIVRFPNITNLCPFGEVFNATRF |
| | ASVYAWNRKRISNCVADYSVLYNSASFSTF |
| | KCYGVSPTKLNDLCFTNVYADSFVIRGDEV |
| | RQIAPGQTGKIADYNYKLPDDFTGCVIAWN |
| | SNNLDSKVGGNYNYLYRLFRKSNLKPFERD |
| | ISTEIYQAGSTPCNGVEGFNCYFPLQSYGF |
| | QPTNGVGYQPYRVVVLSFELLHAPATVCGP |
| | KKSTNLVKNKRVQPTESIVRFPNITNLCPF |
| | GEVFNATRFASVYAWNRKRISNCVADYSVL |
| | YNSASFSTFKCYGVSPTKLNDLCFTNVYAD |
| | SFVIRGDEVRQIAPGQTGKIADYNYKLPDD |
| | FTGCVIAWNSNNLDSKVGGNYNYLYRLFRK |
| | SNLKPFERDISTEIYQAGSTPCNGVEGENC |
| | YFPLQSYGFQPTNGVGYQPYRVVVLSFELL |
| | HAPATVCGPKKSTNLVKNK |

### DETAILED DESCRIPTION OF THE EMBODIMENTS

The present invention discloses a recombinant severe acute respiratory syndrome coronavirus 2 (SARS-CoV-2) RBD trimer protein vaccine capable of generating broad-spectrum cross neutralization activity, and a preparation method and use thereof. Those skilled in the art may make a proper improvement on process parameters for implementation with reference to the content of the specification. It should be particularly noted that all similar substitutes and modifications are apparent to one of skill in the art and are considered to be included within the present invention; and related persons can obviously make a modification or a proper change and combination on the content of this paper without departing from the content, the spirit and the scope of the present invention, so as to implement and apply the technology of the present invention.

In the present invention, unless otherwise specified, scientific and technical terms used herein have the meaning as commonly understood by those skilled in the art. Unless otherwise expressly stated, throughout the specification and claims, the term “including” or its variations such as “including” or “comprising” will be understood as including the stated elements or components, but not excluding other elements or components. Unless otherwise expressly stated, throughout the specification and claims, the term “RBD” represents an RBD structural domain of a spike protein of SARS-CoV-2, which may be understood to be interchangeably with “RBD” or “SARS-CoV-2 RBD region”.

The following makes interpretation for some terms in the present invention.

The term “severe acute respiratory syndrome coronavirus 2”, that is, SARS-CoV-2, belongs to a single-stranded positive strand RNA virus, SARS-like virus species, sarbecovirus subgenus, betacoronavirus, positive coronavirus, coronaviridae, nidovirales, with a cyst membrane. The full length of genome is about 29.9 kb. Most of the genome encodes non-structural proteins and participates in virus replication and translation functions; and a few sequences encode structural proteins, such as a spike protein (S protein), a membrane protein (M protein), a cyst membrane protein (E protein) and a nucleo protein (N protein). In addition, there are several accessory proteins: 3a, 3b, p6, 7a, 7b, 8b, 9b and orf14, and these proteins participate in viral assembly. S, M and E proteins constitute a virus cyst membrane, which is a main surface antigen of the virus causing immune response. The S protein is a transmembrane glycoprotein, has a molecular weight about 150 kDa, and forms a prominent homotrimer on the surface of the virus. The S protein consists of two functional subunits, and is cleaved at a boundary (an S1/S2 cleavage site) between the S1 subunit and the S2 subunit; and the two subunits keep non-covalent binding in conformation before fusion. The S2 subunit is composed of a plurality of structural domains, and has a main function of mediating the fusion between a virus and a host cell. The distal S1 subunit is structurally divided into four different structural domains: a N-terminal structural domain (NTD), a receptor binding structural domain (RBD), a C-terminal structural domain 1 (CTD1) and a C-terminal structural domain 2 (CTD2), where RBD is mainly responsible for binding with a receptor angiotensin converting enzyme 2 (ACE2) on the surface of the host cell, so that the virus is mediated to infect the host cell; therefore, the S protein and RBD are the main targets of the research and development of genetic engineering vaccine at present.

The term “trimer form” is a type in a higher structure of a protein. The protein including three protein subunits is in the trimer form.

The term “at least one” may be understood as that two of three amino acid sequences are the same or the three amino acid sequences are different.

The term “primary structure” is a linear sequence of an amino acid in a peptide or the protein. Conventionally, the primary structure of the protein refers to being from an amino terminal (N terminal) to a carboxy terminal (C terminal).

The term “fusion protein” refers to one, two or more expression products obtained by a DNA recombination technology after genetic recombination. A fusion protein technology is a targeted gene fusion and protein expression method performed for obtaining a great quantity of standard fusion proteins. A novel target protein with a plurality of functions may be constructed and expressed using the fusion protein technology.

The term “vector” is a nucleic acid vehicle, into which polynucleotide may be inserted. If the vector can make the protein, into which polynucleotide is inserted, be expressed, the vector is called an expression vector. The vector may be introduced into the host cell through transformation, transduction or transfection, so that genetic material elements carried by the vector can be expressed in the host cell. The vector is well known to those skilled in the art, including but not limited to: plasmids; phagemids; cosmids; artificial chromosomes, such as yeast artificial chromosome (YAC), bacterial artificial chromosome (BAC) or P1-derived artificial chromosome (PAC); phages such as lambda phages or M13 phages; and animal viruses. Animal viruses that can be used as vectors include, but are not limited to retroviruses (including lentiviruses), adenoviruses, adeno-associated viruses, herpes viruses (such as herpes simplex virus), poxviruses, baculoviruses, papillomaviruses, and papovaviruses (such as SV40). A vector may include a variety of elements that control expression, including, but not limited to promoter sequences, transcription initiation sequences, enhancer sequences, selection elements, and reporter genes. In addition, the vector may further include a replication start site.

The term “host cell” is a cell, into which the nucleic acid molecule has been introduced through a molecular biological technology. These technologies include transfection of the viral vector, transformation with the plasmid vector, and introduction of naked DNA through electroporation, lipofection and particle gun acceleration.

The term “treatment” refers to that the possibility of disease pathologies and occurrence of disease symptoms are reduced, so that, for example, a subject has a longer survival time or reduced discomfort to a certain degree. Treatment may refer to the ability of a therapy reducing the disease symptoms, signs or causes if a therapy is given to the subject. Treatment further refers to relieving or reducing at least on clinical symptom, and/or inhibiting or delaying a progress of a symptom, and/or preventing or delaying the onset of a disease or a condition.

The term “subject” refers to any person or other animals, particularly, other mammals, receiving prevention, treatment and diagnosis. Other mammals may include, for example, a dog, a cat, cattle, horse, sheep, pig, a goat, a rabbit, a rat, a guinea pig, a mouse and the like.

To make those skilled in the art better understand the technical solutions of the present invention, the present invention will be further described in detail below in combination with the specific examples.

### Example 1: Design of SARS-CoV-2 RBD Trimer Protein Based on Protein Structure and Computational Biology

Through comparison and analysis of an SARS-CoV-2 representative mutant strain and a prototype strain (as shown in Table 1), amino acid mutations occurred in a Beta (B.1.351) mutant strain at three sites, that is, K417N, E484K and N501Y; and amino acid mutations occurred in a Kappa (B.1.617.1) mutant strain at two sites, that is, L452R and E484Q. E484K is considered as the most important site mutation leading to immune scape. Through molecular dynamic simulation and free energy calculation, it is found that E484K mutation will significantly reduce the affinity of an RBD and a plurality of neutralizing monoclonal antibodies. In 10 mutant strains that are classified as VOC and VOI and listed by WHO, 6 mutant strains include the E484K mutation. In addition, L452R mutation has been proved to escape the neutralizing antibody and the serum of a recovered COVID-19 patent, and in the ten VOC and VOI mutant strains, three strains include the L452R mutation. Experimental evidence shows that K417N and N501Y mutations can resist some neutralizing antibodies, and in the ten VOC and VOI mutant strains, four strains include the N501Y mutation. In addition, through the evolutionary analysis of 1.2 million known SARS-CoV-2 sequences, it is found that E484K, L452R and N501Y are the most important convergent evolutionary mutation in the RBD. These mutations appear independently in numerous different virus lineages, indicating that these mutations have obvious selection advantages in virus evolution and predicting that these mutations may appear independently or in combination in the future mutant strains. Therefore, it is of great significance to research and develop a COVID-19 vaccine with a broad-spectrum protection ability across epidemic strains.

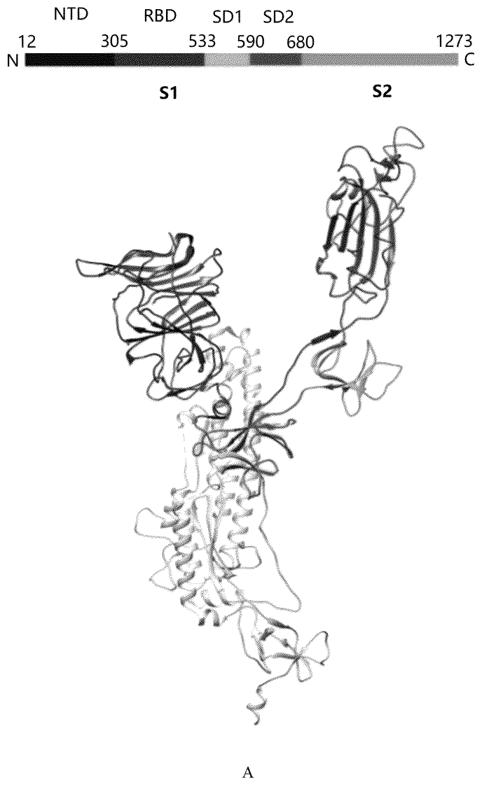

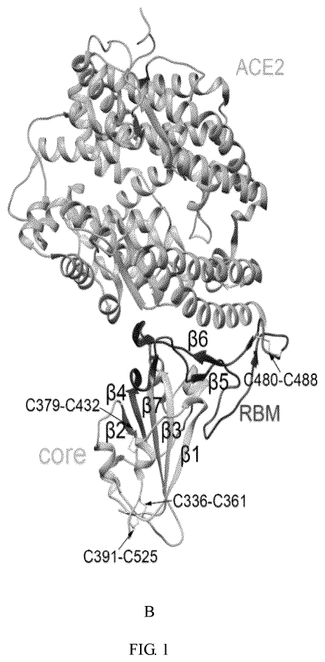

Through analysis on the spatial structure (as shown in FIG. 1) of a natural S protein trimer and calculation on a spatial distance between an N terminal and a C terminal of the RBD structural domain, it is found that trimerization can be realized by the structural feature of the RBD without introducing an exogenous vector or sequence, and a larger spatial barrier is not present. Based on this, SARS-CoV-2 S protein RBD region sequence fragments (the 319th to 537th amino acids) of different mutant strains are intercepted, and the three RBD region fragments are connected end to end in series to form a new fusion protein. The sequences of SARS-CoV-2 prototype strain S protein RBD region fragments (the 319th to 537th amino acids) are shown in SEQ ID NO.1, the sequences of the S protein RBD region fragments (the 319th to 537th amino acids) of the Beta (B.1.351) mutant strain are shown in SEQ ID NO.2, and the sequences of the S protein RBD region fragments (the 319th to 537th amino acids) of the Kappa (B.1.617.1) mutant strain are shown in SEQ ID NO.3.

Through different combinations of SEQ ID NO.1, SEQ ID NO.2 and SEQ ID NO.3, a trimer protein can simultaneously generate the cross neutralizing activity of different SARS-CoV-2 strains to achieve the purpose of researching and developing a broad-spectrum vaccine, which is specifically as follows: a trimer protein A formed by sequentially connecting, SEQ ID NO.1, SEQ ID NO.2 and SEQ ID NO.3, with the amino acid sequence shown in SEQ ID NO.4; a trimer protein B formed by sequentially connecting, SEQ ID NO.1, SEQ ID NO.2 and SEQ ID NO.1, with the amino acid sequence shown in SEQ ID NO.5; a trimer protein C formed by sequentially connecting, SEQ ID NO.1, SEQ ID NO.2 and SEQ ID NO.2, with the amino acid sequence shown in SEQ ID NO.6; a trimer protein D formed by sequentially connecting, SEQ ID NO.2, SEQ ID NO.1 and SEQ ID NO.2, with the amino acid sequence shown in SEQ ID NO.7; and a trimer protein E formed by sequentially connecting, SEQ ID NO.2, SEQ ID NO.2 and SEQ ID NO.2, with the amino acid sequence shown in SEQ ID NO.8.

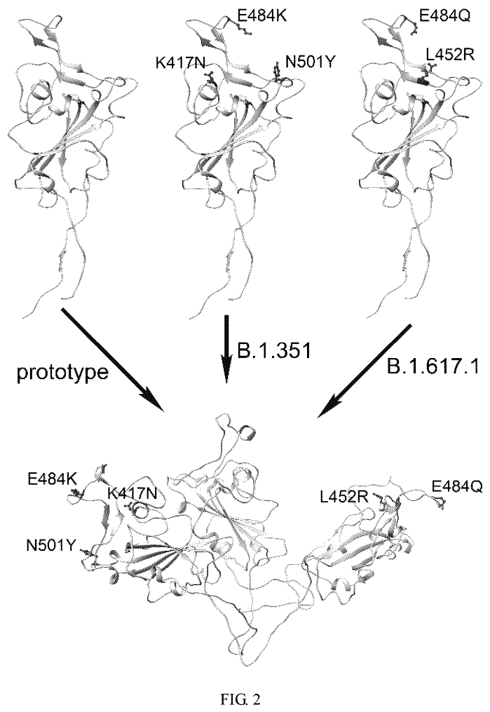

Taking the trimer protein A (with the amino acid sequence shown in SEQ ID NO.4) as an example, a possible spatial structure is constructed by homologous modeling. The result is shown in FIG. 2 and shows that the fusion protein includes three independent RBD structural domains, a trimer form with stable antigen conformation may be formed, and the protein covers a key active mutation site and a potential immune escape site. It is theoretically speculated that the recombinant vaccine taking the sites as target antigen has the broad-spectrum protection ability across epidemic strains. In addition, compared with the traditional strategy of preparing a plurality of monovalent vaccines to realize the broad-spectrum protection through multivalent protection, multivalent broad-spectrum protective effect is achieved on an antigen molecule, and the advantages in the time cost, economic cost and vaccine productivity of vaccine preparation are significant.

TABLE 1
Mutation of SARS-CoV-2 Representative
Epidemic Strain RBD Region

| WHO | Mutant Strain | VOC/ | Discovery | Mutation of RBD |
|---|---|---|---|---|
| Name | Lineage | VOI | Time | Region |
| Alpha | B.1.1.7 | VOC | September | N501Y |
| | | | 2020 | |
| Beta | B.1.351 | VOC | May 2020 | K417N, E484K, |
| | | | | N501Y |
| Gamma | P.1 | VOC | November | K417T, E484K, |
| | | | 2020 | N501Y |
| Delta | B.1.617.2 | VOC | November | L452R, T478K |
| | | | 2020 | |
| Epsilon | B.1.427/B.1.429 | VOI | March 2020 | L452R |
| Zeta | P.2 | VOI | April 2020 | E484K |
| Eta | B.1.525 | VOI | December | E484K |
| | | | 2020 | |
| Theta | P.3 | VOI | January | E484K, N501Y |
| | | | 2021 | |
| Iota | B.1.526 | VOI | November | E484K |
| | | | 2020 | |
| Kappa | B.1.617.1 | VOI | October | L452R, E484Q |
| | | | 2020 | |

### Example 2: Expression, Purification and Identification of Trimer Protein

According to codon preference of a CHO cell expression system, codon optimization was performed on a nucleotide sequence encoding a protein A to a protein E (with amino acid sequences shown in SEQ ID NO.4 to SEQ ID NO.8). The optimized nucleotide sequence of the protein A (with the amino acid sequence shown in SEQ ID NO.4) is shown in SEQ ID NO.9, the optimized nucleotide sequence of the protein B (with the amino acid sequence shown in SEQ ID NO.5) is shown in SEQ ID NO.10, the optimized nucleotide sequence of the protein C (with the amino acid sequence shown in SEQ ID NO.6) is shown in SEQ ID NO.11, SEQ ID NO.12, SEQ ID NO.13 or SEQ ID NO.14, the optimized nucleotide sequence of the protein D (with the amino acid sequence shown in SEQ ID NO.7) is shown in SEQ ID NO.15, and the optimized nucleotide sequence of the protein E (with the amino acid sequence shown in SEQ ID NO.8) is shown in SEQ ID NO.16 or SEQ ID NO.17.

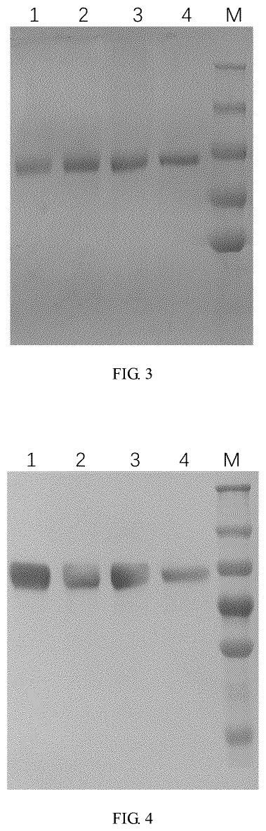

After being constructed, a CHO cell expression vector was transfected into a 293FT cell or a CHO cell to construct a recombinant cell line. A cell line capable of stably secreting and expressing the RBD trimer protein was screened by a limited dilution method. Finally, the protein A, the protein B and the protein C were expressed successfully. Through a series of chromatographic purification, the protein A, the protein B and the protein C obtain trimer proteins with the purity greater than or equal to 95%. The expression quantities of the protein D and the protein E are excessively low, and the target protein with higher purity is not obtained after purification. The SDS-PAGE detection result of the protein A, the protein B and the protein C is shown in FIG. 3. The molecular weight of the protein is 70 to 100 kD; and some substances, such as a dimer protein and a monomer protein, related to the product is visible.

The purified protein A, protein B and protein C were electrotransferred onto a polyvinylidene fluoride (PVDF) membrane after being subjected to SDS-PAGE electrophoresis, and then were subjected to Western-blot identification using an RBD-specific antibody (manufacturer: Beijing Sino Biological Ltd. Co.; article number: 40591-T62; dilutability: 2000 folds) (the result is shown in FIG. 4). It shows that all the proteins may bind to the RBD-specific antibody, and has good biological activity. The purified protein A, protein B and protein C were subjected to molecular-exclusion chromatography analysis by a TSKgel G2500PW gel chromatographic column, and the purity of all the purified proteins is greater than 90%.

### Example 3: Biological Analysis on Binding of Neutralizing Monoclonal Antibody

The purified trimer protein A, protein B and protein C, a trimer protein (obtained by performing 293FT cell or CHO cell recombinant expression and chromatographic purification on proteins, with the amino acid sequence shown in SEQ ID NO.18, which are formed by sequentially connecting three amino acid fragments shown in SEQ ID NO.1), a dimer protein (obtained by performing 293FT cell or CHO cell recombinant expression and chromatographic purification on proteins, with the amino acid sequence shown in SEQ ID NO.19, which are formed by sequentially connecting two amino acid fragments shown in SEQ ID NO.1), an RBD protein (manufacturer: Beijing Sino Biological Ltd. Co.; article number: 40592-V08B), an RBD protein (K417N, E484K and N501Y; manufacturer: Beijing Sino Biological Ltd. Co.; article number: 40592-V08H85) consistent with the virus mutation sites of Beta (B.1.351) strain, and an RBD protein (L452R and E484Q; manufacturer: Beijing Sino Biological Ltd. Co.; article number: 40592-V08H85) consistent with the virus mutation sites of Kappa (B.1.617.1) strain were diluted with coating liquid to 4 μg/ml, 2 μg/ml, 1 μg/ml, 0.5 μg/ml, 0.25 μg/ml, 0.125 μg/ml, 0.0625 μg/ml, 0.03125 μg/ml, 0.015625 μg/ml, 0.007813 μg/ml, 0.003906 μg/ml, 0.001953 μg/ml, and were coated to a 96-well ELISA plate by 100 μl/well at 4° C. for 8 to 12 h. and a blank well served as negative control; after the plate was washed with a PBST solution, a confining liquid was added for confining at 37° C. for 3 h; after the plate was washed by the PBST solution, a diluted MM43 monoclonal antibody (manufacturer: Beijing Sino Biological Ltd. Co.; article number: 40591-MM43; dilutability: 2000 folds) or a diluted MM57 monoclonal antibody (manufacturer: Beijing Sino Biological Ltd. Co.; article number: 40592-MM57; dilutability: 2000 folds) or a diluted R001 monoclonal antibody (manufacturer: Beijing Sino Biological Ltd. Co.; article number: 40592-R001; dilutability: 2000 folds) or a diluted R117 monoclonal antibody (manufacturer: Beijing Sino Biological Ltd. Co.; article number: 40592-R117; dilutability: 2000 folds) were respectively added in 100 μl/well for incubation at 37° C. for 1 h; after the plate was washed with the PBST solution, a diluted horse radish peroxidase-labeled goat anti-mouse or goat anti-rabbit antibody was added in 100 μl/well for incubation at 37° C. for 1 h; after the plate was washed with the PBST solution, chromogenic solutions A and B were added sequentially for developing for 5 to 10 min at room temperature, and a stop solution C was added; and values were read from a microplate reader at double wavelengths (OD450 nm and 630 nm) to determine a cutoff value, and a curve of protein concentration-absorbance value was drawn.

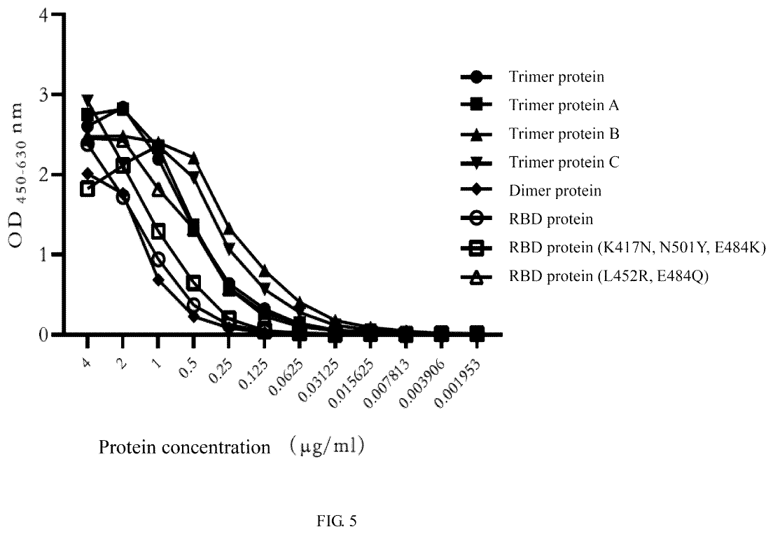

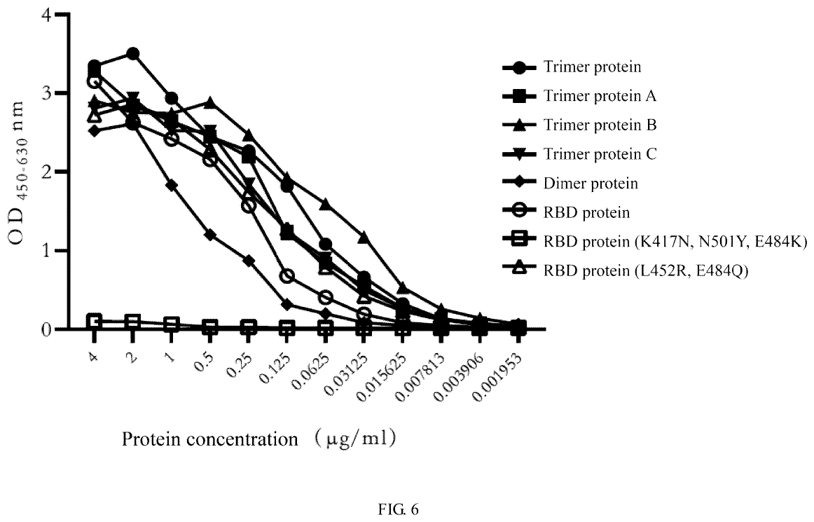

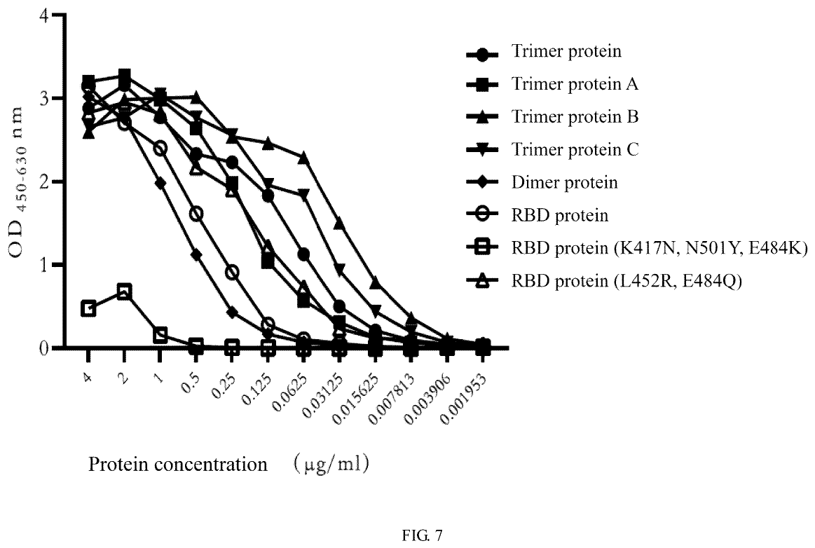

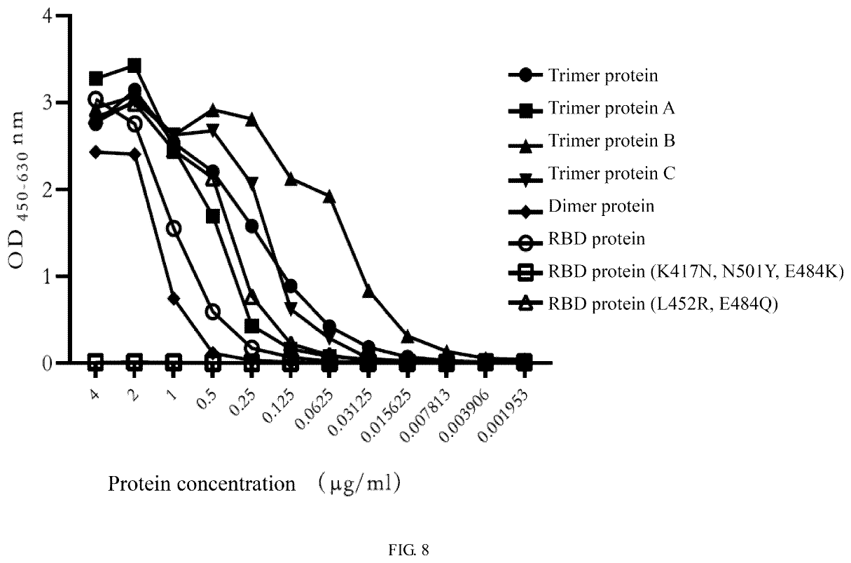

The result of the binding activity with the MM43 monoclonal antibody is shown in FIG. 5, the result of the binding activity with the MM57 monoclonal antibody is shown in FIG. 6, the result of the binding activity with the R001 monoclonal antibody is shown in FIG. 7, and the result of the binding activity with the R117 monoclonal antibody is shown in FIG. 8. It can be seen from the results that the trimer protein A is bound to all the neutralizing monoclonal antibodies to varying degrees, where the binding activity with the MM43 antibody is basically consistent with the trimer protein, and the binding activity of the MM57 and R117 monoclonal antibodies is lower than the trimer protein, indicating that the trimer protein has the RBD protein property of the prototype strain, and also has the RBD protein characteristic of the Beta (B.1.351) strain.

### Example 4: Physical and Chemical Properties and Activity Detection of Trimer Protein A

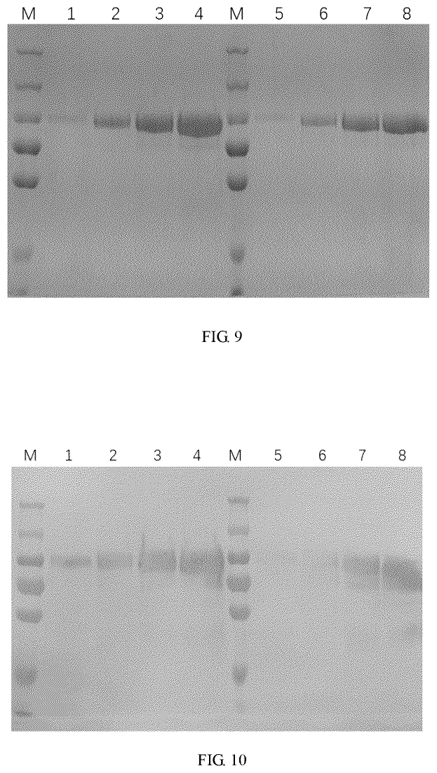

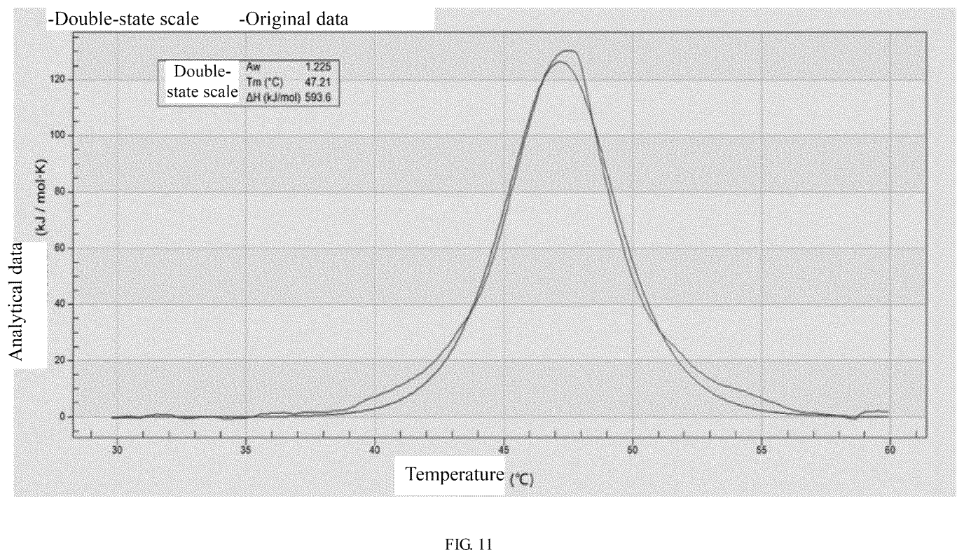

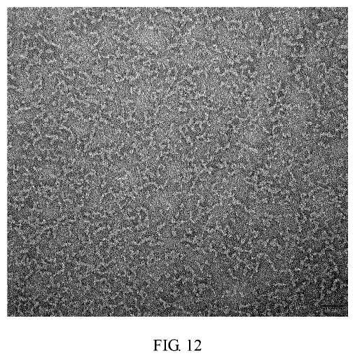

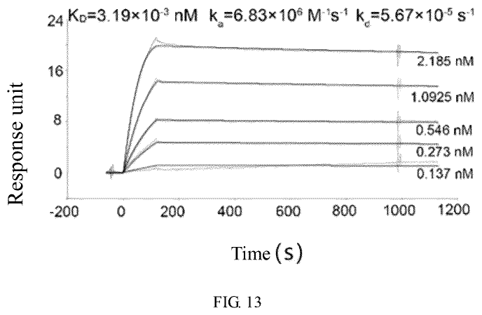

A primary sequence of a trimer protein A includes RBD region amino acid sequences of a prototype strain, a Beta (B.1.351) mutant strain and a Kappa (B.1.617.1) mutant strain, and is a mutations-integrated trimeric form of RBD (mutI tri-RBD). The purified trimer protein A was further analyzed. The results are shown in FIG. 9 and FIG. 10 through SDS-PAGE detection and Western-blot analysis and by taking the trimer protein as control. It can be seen that the molecular weight of the trimer protein A is basically the same as that of the trimer protein and between 70 and 100 kD. The integrated molecular weight of the protein is detected by an MALDI-TOF MS method. The molecular weight of the trimer protein A is about 87.836 kD and is slightly greater than a theoretical value (74 kD), which is related to protein glycosylation modification. The protein glycosylation modification is detected by an UPLC-MS method. The result shows that the recombinant expressed trimer protein A has glycosylation modification with many sites. The trimer protein A was subjected to secondary structure detection by a circular dichroism spectrum. The result shows that the a spiral ratio is 13.5%, the p folding ratio is 23.3%, the R corner ratio is 11.4%, and the other is 51.8%, which keeps basically consistent with the theoretical value. The formation of a disulfide bond was detected by an UPLC-MS method. The result also shows that each RBD monomer in the trimer protein A can form four pairs of disulfide bonds, which is consistent with the pairing result of the disulfide bonds in the RBD natural structure. The heat stability of the trimer protein A was detected by a differential scanning calorimetry The result is shown in FIG. 11. Tm value is 47.2° C., and the trimer protein A has high heat stability. The form of the protein was observed by a transmission electron microscope. The result is shown in FIG. 12. It can be seen that the grain diameter of the trimer protein A is small and about 3 to 5 nm, but a clearer morphological structure image is not observed. In addition, the affinity of the trimer protein A and the hACE receptor protein was detected by a surface plasmon resonance technology. The result is shown in FIG. 13. KD value is 3.19×10−3 nM through calculation. The trimer protein A and the hACE receptor protein have high affinity, which proves that the trimer protein A has high biological activity.

### Example 5: Preparation of Recombinant SARS-CoV-2 Vaccine

The purified protein was diluted to twice a target antigen concentration and was mixed with an aluminium hydroxide adjuvant of 1.2 mg/ml in a ratio of 1:1 (w/w) for adsorption; a mixture was stirred by a magnetic stirrer for 40-120 min at a rotating speed of 200-300 rpm to obtain a semi-finished vaccine, where the content of a residual protein in a supernatant should be less than 10% of a total protein content; and the semi-finished vaccine was aseptically dispensed into vials by 0.5 ml per vial, so as to obtain finished vaccines.

### Example 6: Evaluation on Immunological Effect of Recombinant SARS-CoV-2 Vaccine

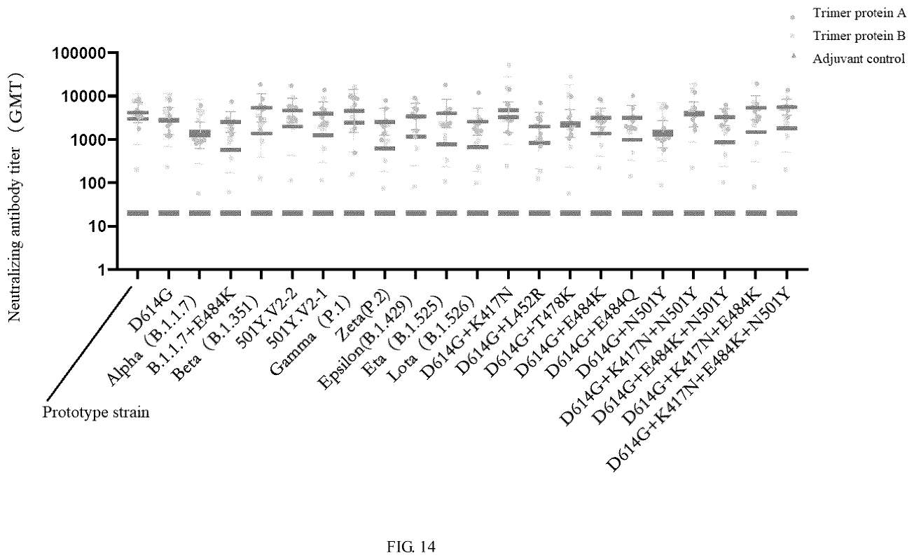

The prepared vaccines (such as the trimer protein A or trimer protein) were respectively intraperitoneally injected for immunizing BALB/c mice (purchased from Beijing Vitalriver Experimental Animal Technology Ltd. Co., SPF grade, female, 6 to 8 weeks old), 2 μg/dose/mouse, respectively immunizing two needles at 0 w and 3 w, sampling blood at 5 w, and separating serum. The neutralizing activity of the immunized mouse serum for various pseudoviruses (as shown in Table 2) was detected by a pseudovirus trace neutralization test. The result is shown in FIG. 14, and the serum antibody GMT value is shown in Table 3. It can be seen that the trimer protein A can generate the neutralizing activity for various pseudoviruses, has a certain protection potential for the mutant strains causing immune escape and the mutant strains that may appear in the future, and is expected to generate a broad-spectrum protection ability.

TABLE 2
SARS-CoV-2 Pseudovirus Information List

| | | Mutation of RBD |
|---|---|---|
| Number | Pseudovirus Name | Region Site |
| 1 | Prototype strain | None |
| 2 | D614G | None |
| 3 | Alpha (B.1.1.7) | N501Y |
| 4 | B.1.1.7 + E484K | N501Y, E484K |
| 5 | Beta (B.1.351) | K417N, E484K, N501Y |
| 6 | 501Y.V2-2 | N501Y |
| 7 | 501Y.V2-1 | N501Y |
| 8 | Gamma (P.1) | K417T, E484K, N501Y |
| 9 | Zeta (P.2) | E484K |
| 10 | Epsilon (B.1.429) | L452R |
| 11 | Eta (B.1.525) | E484K |
| 12 | Lota (B.1.526) | E484K |
| 13 | D614G + K417N | K417N |
| 14 | D614G + L452R | L452R |
| 15 | D614G + T478K | T478K |
| 16 | D614G + E484K | E484K |
| 17 | D614G + E484Q | E484Q |
| 18 | D614G + N501Y | N501Y |
| 19 | D614G + K417N + N501Y | K417N, N501Y |
| 20 | D614G + E484K + N501Y | E484K, N501Y |
| 21 | D614G + K417N + E484K | K417N, E484K |
| 22 | D614G + K417N + E484K + N501Y | K417N, E484K, N501Y |

TABLE 3
Neutralizing Antibody GMT Value (Pseudovirus
Trace Neutralization Test)

| | | Neutralizing Antibody | |
|---|---|---|---|
| | | GMT Value | |
| | | Trimer | Trimer |
| Number | Pseudovirus Name | protein A | protein |
| 1 | Prototype strain | 4152 | 2970 |
| 2 | D614G | 2680 | 2851 |
| 3 | Alpha (B.1.1.7) | 1246 | 1532 |
| 4 | B.1.1.7 + E484K | 2533 | 569 |
| 5 | Beta (B.1.351) | 5377 | 1369 |
| 6 | 501Y.V2-2 | 4627 | 1980 |
| 7 | 501Y.V2-1 | 3911 | 1259 |
| 8 | Gamma (P.1) | 4546 | 2451 |
| 9 | Zeta (P.2) | 2548 | 622 |
| 10 | Epsilon (B.1.429) | 3388 | 1157 |
| 11 | Eta (B.1.525) | 4006 | 764 |
| 12 | Lota (B.1.526) | 2585 | 666 |
| 13 | D614G + K417N | 3299 | 4715 |
| 14 | D614G + L452R | 1982 | 832 |
| 15 | D614G + T478K | 2340 | 2074 |
| 16 | D614G + E484K | 3188 | 1368 |
| 17 | D614G + E484Q | 3176 | 984 |
| 18 | D614G + N501Y | 1303 | 1506 |
| 19 | D614G + K417N + N501Y | 3726 | 4092 |
| 20 | D614G + E484K + N501Y | 3233 | 872 |
| 21 | D614G + K417N + E484K | 5382 | 1468 |
| 22 | D614G + K417N + E484K + | 5616 | 1803 |
| | N501Y | | |

A pseudovirus trace neutralization test: the neutralizing antibody titer of the immunized serum for various pseudoviruses (as shown in Table 2) was detected by the pseudovirus trace neutralization test, the pseudovirus reporter gene was firefly luciferase, the pseudovirus working concentration is (1−2)×104 TCID50/ml, and the dilution ratio of the serum at 50% infection inhibition rate was calculated by a Reed-Muench method, that is the neutralizing antibody titer of the serum sample.

The above are preferred embodiments of the present invention, and it should be noted that, for those of ordinary skill in the art, several improvements and modifications may be made without departing from the principle of the present invention, and the improvements and modifications are also regarded to be within the protection scope of the present invention.

1: A recombinant SARS-CoV-2 RBD trimer protein capable of generating broad-spectrum cross neutralization activity, wherein the trimer protein is composed of subunits of three SARS-CoV-2 protein RBD regions, and the amino acid sequences of the trimer protein is amino acid sequence shown in SEQ ID NO. 4.

2. (canceled)

3. (canceled)

4. (canceled)

5. (canceled)

6. (canceled)

7. (canceled)

8. (canceled)

9. (canceled)

10: A fusion protein, wherein the amino acid sequences of the fusion protein comprise amino acid sequences of the recombinant SARS-CoV-2 RBD trimer protein according to claim 1.

11: The fusion protein according to claim 10, further comprising one or more of signal peptides, tags or immune-enhancing peptides.

12: A nucleic acid molecule, comprising a nucleotide sequence encoding the recombinant SARS-CoV-2 RBD trimer protein according to claim 1.

13. (canceled)

14. (canceled)

15. (canceled)

16: The nucleic acid molecule according to claim 12, wherein the nucleotide sequences of the nucleic acid molecule is nucleotide sequences shown in SEQ ID NO.9.

17. (canceled)

18: A vector, comprising the nucleic acid molecule according to claim 12.

19: A host cell, comprising the nucleic acid molecule according to claim 12.

20: A host cell, comprising the vector according to claim 18.

21: The host cell according to claim 20, wherein the host cell is Escherichia coli, a yeast cell, an insect cell or a mammalian cell.

22: The host cell according to claim 21, wherein the host cell is a CHO cell.

23: A method for preparing the recombinant SARS-CoV-2 RBD trimer protein according to claim 1, comprising the following steps:
step A): preparing a nucleic acid molecule comprising a nucleotide sequence encoding the recombinant SARS-CoV-2 RBD trimer protein according to claim 1, constructing an expression vector of the nucleic acid molecule, and transforming or transfecting the expression vector into the host cell;
step B): performing protein expression by using a product in step A); and
step C): purifying an expression product obtained in step B) to obtain the recombinant SARS-CoV-2 RBD trimer protein.

24. (canceled)

25: Use of the recombinant SARS-CoV-2 RBD trimer protein according to claim 1 to preparation of a drug for treating and/or preventing SARS-CoV-2 infection and/or diseases caused by SARS-CoV-2.

26. (canceled)

27. (canceled)

28. (canceled)

29. (canceled)

30. (canceled)

31. (canceled)

32: A recombinant protein vaccine, comprising the recombinant SARS-CoV-2 RBD trimer protein according to claim 1, and an adjuvant.

33. (canceled)

34. (canceled)

35. (canceled)

36: The recombinant protein vaccine according to claim 32, wherein the adjuvant is aluminium hydroxide, aluminium phosphate, MF59 or CpG.

37: The recombinant protein vaccine according to claim 36, wherein the adjuvant is aluminium hydroxide.

38: A method for preparing the recombinant protein vaccine according to claim 32, wherein the recombinant SARS-CoV-2 RBD trimer protein obtained through purification and the adjuvant are mixed.

39: A genetic engineering vector vaccine, comprising the nucleic acid molecule according to claim 12.

40: A nucleic acid vaccine, comprising the nucleic acid molecule according to claim 1.

41. (canceled)

42. (canceled)

43. (canceled)

44. (canceled)

45. (canceled)

46. (canceled)

47: A drug composition, comprising the vaccine according to claim 32, and a pharmaceutically acceptable vector.

48. (canceled)

49: A drug composition, comprising the vaccine according to claim 39, and a pharmaceutically acceptable vector.

50. (canceled)

51. (canceled)

52. (canceled)

53. (canceled)

54: A drug composition, comprising the vaccine according to claim 40, and a pharmaceutically acceptable vector.


SEQUENCE LISTING (19 sequences)

SEQ ID NO: 1 - protein, 219 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 2 - protein, 219 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 3 - protein, 219 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNK

SEQ ID NO: 4 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNK

SEQ ID NO: 5 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 6 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 7 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 8 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNK

SEQ ID NO: 9 - DNA, 1977 bp, Artificial Sequence
  SARS-CoV-2
atgagagtgcagcctaccgagagcattgtcagattccctaacatcaccaatctgtgccctttcggcgaggtgttcaacgctacaagatttgctagcgtgtacgcttggaacagaaagagaatcagcaattgcgtcgccgactacagcgtgctgtacaacagcgcctccttcagcaccttcaagtgctacggcgtcagccctacaaaactgaatgacctgtgctttacaaatgtgtatgccgatagcttcgtcatcagaggcgacgaggtgaggcaaatcgctcctggccaaaccggcaagatcgccgactacaattacaagctgcctgacgatttcaccggatgcgtgattgcctggaacagcaacaacctggacagcaaggtgggcggcaactacaactacctgtacagactgtttagaaaaagcaacctgaagcctttcgagagagacatcagcaccgagatctaccaagccggcagcacaccttgcaacggcgtggagggattcaactgctatttccctctgcagagctacggctttcagcctaccaacggcgtgggctatcagccttatagagtcgtggtgctcagcttcgaactcctgcacgcccctgctacagtgtgtggccctaagaagtccaccaacctggtgaagaataagagagtgcaacctaccgagagcatcgtgagattccccaatattaccaacctgtgcccctttggcgaagtgttcaacgccacaagattcgctagcgtgtacgcctggaacagaaagagaatcagcaactgcgtggccgactacagcgtgctctacaactccgctagcttcagcacctttaagtgctacggcgtgagccctaccaagctgaacgacctgtgctttaccaacgtctatgctgacagctttgtgatcagaggcgacgaggtgagacagatcgctcccggacagaccggcaatatcgccgattacaactacaagctgcccgacgatttcaccggatgcgtgatcgcctggaacagcaacaatctggactccaaggtgggaggcaattacaattacctgtacagactgtttagaaaatccaacctgaagcctttcgagagagacatttccaccgagatctaccaagccggctccacaccttgcaatggagtgaagggcttcaactgctacttccctctgcagagctacggctttcagcctacatacggagtcggctatcagccttacagagtcgtcgtcctgagcttcgagctcctgcacgcccccgccaccgtctgcggacctaagaagtccacaaacctcgtcaaaaacaagagagtgcagcctaccgagagcatcgtgaggtttcctaacatcaccaacctgtgccctttcggcgaggtctttaacgccacaagattcgctagcgtctacgcctggaacagaaagaggattagcaattgtgtcgccgactacagcgtgctgtacaacagcgctagcttcagcaccttcaagtgctacggcgtgagccctaccaagctgaacgacctgtgctttacaaacgtgtatgccgacagcttcgtgattaggggcgacgaggtgaggcaaatcgctcctggccaaaccggcaagatcgccgactacaactacaagctgcctgacgatttcaccggctgcgtgatcgcctggaacagcaacaacctggatagcaaggtgggcggcaattacaactacagatatagactgttcagaaagagcaacctgaagcctttcgagagagacatctccaccgagatctatcaagccggcagcacaccttgcaacggagtgcaaggcttcaactgctatttccctctgcaatcctacggctttcagcctaccaacggagtgggctatcagccttacagagtcgtcgtgctgagcttcgagctgctgcacgcccctgccacagtgtgcggacctaaaaagagcacaaatctggtgaagaacaagtga

SEQ ID NO: 10 - DNA, 1977 bp, Artificial Sequence
  SARS-CoV-2
atgagagtgcagcctacagagagcattgtgagattccctaacatcacaaacctgtgccctttcggcgaggtgttcaacgccacaagattcgctagcgtgtacgcctggaacagaaaaagaattagcaattgcgtcgccgattacagcgtgctgtataacagcgcttccttcagcaccttcaagtgctacggcgtcagccctacaaagctgaacgacctgtgcttcacaaacgtgtatgccgacagcttcgtgatcagaggcgacgaggtgaggcaaatcgctcctggccaaaccggcaagatcgccgactacaattacaagctgcctgatgacttcaccggatgcgtgattgcctggaacagcaacaacctggacagcaaagtcggaggaaactacaactacctgtacagactgttcagaaagagcaacctgaagcctttcgagagagacatcagcaccgagatctaccaagccggctccacaccttgcaacggcgtggagggattcaactgctacttccctctgcagagctacggctttcagcccaccaacggcgtgggctatcagccttacagagtggtcgtcctgagctttgagctgctccacgcccctgccaccgtctgcggccccaaaaaaagcaccaatctggtgaagaacaagagggtgcagcctacagagagcatcgtgagattccctaacattaccaacctgtgccctttcggagaagtgtttaacgccacaagattcgcctccgtctacgcttggaatagaaagaggatcagcaactgcgtggccgactacagcgtgctgtacaattccgcctccttcagcaccttcaagtgttacggcgtgagccctaccaagctgaacgacctgtgcttcaccaacgtgtacgccgatagcttcgtgattagaggcgacgaggtgaggcagatcgcccctggacaaaccggcaacattgctgactacaattacaagctgcctgacgacttcaccggctgcgtgatcgcctggaacagcaacaacctggacagcaaggtcggaggcaattacaattacctctatagactgttcagaaaaagcaatctcaagcctttcgaaagagacattagcaccgagatctaccaagccggcagcaccccctgcaacggcgtgaaaggattcaactgctacttccccctgcagagctacggctttcagcccacctacggagtgggctatcaaccctacagagtggtcgtgctctccttcgagctgctgcatgcccctgccacagtgtgcggacctaaaaagtccaccaacctcgtgaagaacaagagagtgcagcctacagagagcatcgtgaggttccccaacatcaccaacctgtgccctttcggcgaggtgtttaacgctacaagattcgctagcgtgtacgcttggaacagaaagagaatctccaattgcgtggccgactacagcgtgctgtacaacagcgctagcttcagcaccttcaagtgctacggagtgtcccctaccaagctgaacgacctgtgcttcaccaacgtgtacgccgatagcttcgtgattagaggagacgaagtgagacagattgctcctggacagaccggcaagatcgccgactacaattacaagctgcctgatgactttaccggctgtgtgattgcctggaacagcaacaacctggacagcaaggtgggcggcaactacaactatctgtacagactcttcagaaagagcaatctgaagccttttgaaagggacatcagcaccgagatctatcaagccggcagcaccccttgcaacggagtcgagggcttcaactgctactttcctctgcagagctatggctttcagcctaccaacggcgtcggatatcagccttacagagtcgtggtgctgagcttcgagctgctccacgcccctgccaccgtctgcggccctaagaaaagcaccaacctggtcaagaacaaatga

SEQ ID NO: 11 - DNA, 1977 bp, Artificial Sequence
  SARS-CoV-2
atgagagtgcagcctacagagagcattgtgagattccctaacatcaccaatctgtgccctttcggcgaggtgttcaacgccacaagattcgctagcgtgtacgcctggaacagaaagaggatctccaattgcgtggctgactatagcgtgctgtacaatagcgctagcttcagcaccttcaagtgctacggcgtcagccctacaaagctgaacgacctgtgcttcacaaacgtgtatgccgacagcttcgtgatcagaggcgacgaggtgaggcaaatcgctcctggccaaaccggcaagatcgccgactataattataagctgcctgatgacttcaccggctgcgtcatcgcctggaacagcaataatctggacagcaaggtcggaggcaactacaactacctgtacagactgttcagaaagagcaacctgaagcctttcgagagagacatcagcaccgagatttaccaagccggctccaccccttgcaacggcgtggaaggcttcaactgctacttccctctgcagtcctacggctttcagcctacaaacggcgtgggctaccaaccctacagagtcgtggtgctcagcttcgagctgctgcatgcccctgctaccgtgtgcggccccaaaaagagcaccaacctcgtgaagaataagagagtgcagcctaccgagagcatcgtcagattccctaacatcaccaatctctgccccttcggcgaggtgttcaacgccacaagattcgctagcgtctacgcttggaatagaaagagaatcagcaactgtgtggccgactatagcgtgctgtacaacagcgctagctttagcacctttaagtgctacggcgtgtcccctaccaagctgaacgacctgtgcttcacaaatgtgtacgccgacagcttcgtgatcagaggagacgaggtgaggcagatcgcccctggacaaaccggcaatattgccgactacaactacaagctgcccgacgacttcaccggctgcgtgatcgcctggaacagcaacaacctcgacagcaaagtgggaggcaattacaactacctgtatagactgtttagaaagagcaacctgaagcctttcgagagggacatctccaccgagatctaccaagccggcagcaccccttgtaacggcgtgaagggcttcaactgttacttccctctgcagagctatggctttcagcctacatacggagtgggctatcagccttacagagtggtggtcctctcctttgaactcctgcatgcccctgccacagtgtgcggacctaaaaagagcaccaacctcgtgaagaataagagagtgcagcctaccgagagcatcgtgagattccccaatatcaccaacctgtgtcctttcggcgaggtgttcaatgccacaagattcgctagcgtctatgcctggaacagaaagagaatctccaattgcgtggccgactacagcgtgctgtacaacagcgctagcttcagcaccttcaagtgctacggagtgagccctaccaagctgaacgacctctgctttaccaatgtgtacgccgatagcttcgtcattagaggagacgaggtgagacagattgctcctggacagaccggcaacatcgccgactacaactacaagctgcctgacgattttaccggctgtgtgatcgcctggaacagcaacaacctggacagcaaggtcggcggcaactacaactatctgtacagactgtttagaaagagcaacctgaagcctttcgaaagggacatcagcaccgagatctatcaagccggctccaccccttgcaacggcgtcaagggctttaactgctacttccctctgcagagctacggctttcagcctacctacggcgtcggatatcagccttatagagtggtcgtgctgagcttcgagctgctccacgcccctgccaccgtctgcggccctaaaaagagcaccaacctggtcaagaacaaatga

SEQ ID NO: 12 - DNA, 1977 bp, Artificial Sequence
  SARS-CoV-2
atgagagtgcagcccacagagagcattgtgagattccccaacatcaccaatctgtgccccttcggcgaggtgttcaacgccacaagattcgctagcgtgtacgcctggaacagaaagcggatctccaattgcgtggctgactatagcgtgctgtacaatagcgctagcttcagcaccttcaagtgctacggcgtcagccccacaaagctgaacgacctgtgcttcacaaacgtgtatgccgacagcttcgtgatcagaggcgacgaggtgcggcaaatcgctcccggccaaaccggcaagatcgccgactataattataagctgcccgatgacttcaccggctgcgtcatcgcctggaacagcaataatctggacagcaaggtcgggggcaactacaactacctgtacagactgttcagaaagagcaacctgaagcccttcgagagagacatcagcaccgagatttaccaagccggctccaccccctgcaacggcgtggaaggcttcaactgctacttccccctgcagtcctacggctttcagcccacaaacggcgtgggctaccaaccttacagagtcgtggtgctcagcttcgagctgctgcatgcccccgctaccgtgtgcggccctaaaaagagcaccaacctcgtgaagaataagagagtgcagcccaccgagagcatcgtcagattccccaacatcaccaatctctgccctttcggcgaggtgttcaacgccacaagattcgctagcgtctacgcttggaaccggaagagaatcagcaactgtgtggccgactatagcgtgctgtacaacagcgctagctttagcacctttaagtgctacggcgtgtcccccaccaagctgaacgacctgtgcttcacaaatgtgtacgccgacagcttcgtgatcagaggggacgaggtgcggcagatcgcccccgggcaaaccggcaatattgccgactacaactacaagctgcctgacgacttcaccggctgcgtgatcgcctggaacagcaacaacctcgacagcaaagtggggggcaattacaactacctgtatagactgtttagaaagagcaacctgaagcccttcgagcgggacatctccaccgagatctaccaagccggcagcaccccctgtaacggcgtgaagggcttcaactgttacttccccctgcagagctatggctttcagcccacatacggggtgggctatcagccctacagagtggtggtcctctcctttgaactcctgcatgcccccgccacagtgtgcgggcccaaaaagagcaccaacctcgtgaagaataagagagtgcagcccaccgagagcatcgtgagattccctaatatcaccaacctgtgtcccttcggcgaggtgttcaatgccacaagattcgctagcgtctatgcctggaacagaaagagaatctccaattgcgtggccgactacagcgtgctgtacaacagcgctagcttcagcaccttcaagtgctacggggtgagccccaccaagctgaacgacctctgctttaccaatgtgtacgccgatagcttcgtcatccggggggacgaggtgagacagattgctcccgggcagaccggcaacatcgccgactacaactacaagctgcccgacgattttaccggctgtgtgatcgcctggaacagcaacaacctggacagcaaggtcggcggcaactacaactatctgtacagactgtttagaaagagcaacctgaagcccttcgaacgggacatcagcaccgagatctatcaagccggctccaccccctgcaacggcgtcaagggctttaactgctacttccccctgcagagctacggctttcagcccacctacggcgtcgggtatcagccctaccgggtggtcgtgctgagcttcgagctgctccacgcccccgccaccgtctgcggccccaaaaagagcaccaacctggtcaagaacaaatga

SEQ ID NO: 13 - DNA, 1977 bp, Artificial Sequence
  SARS-CoV-2
atgagagtgcagcctaccgagagcatcgtgagatttcctaatatcaccaatctctgccccttcggcgaggtgtttaacgccacaagatttgctagcgtgtacgcctggaacagaaaaagaatctccaactgcgtggccgactacagcgtcctgtacaatagcgctagcttcagcaccttcaagtgctacggcgtctcccctacaaagctgaatgacctgtgttttaccaacgtgtatgccgacagcttcgtgattagaggcgatgaggtgaggcagatcgcccctggacagaccggcaagatcgctgattacaactacaagctgcctgacgacttcaccggctgcgtgatcgcctggaacagcaacaacctggatagcaaggtgggcggcaactacaactatctgtacagactgttcagaaaaagcaacctgaagccttttgagagagacatcagcacagagatctaccaagccggcagcaccccttgcaacggcgtggaaggatttaactgctatttccctctgcagagctatggcttccaacctaccaacggagtgggctatcagccctacagagtggtcgtgctgagcttcgagctgctgcacgctcctgccaccgtgtgcggccccaaaaagtccaccaacctggtgaagaataaaagagtgcagcctaccgaatccatcgtgagattccctaacatcaccaacctgtgccctttcggcgaggtcttcaacgccacaagatttgctagcgtgtacgcctggaacagaaagagaatcagcaactgcgtggccgactacagcgtcctgtacaacagcgcctccttcagcaccttcaaatgctacggcgtgtcccctaccaagctgaatgacctgtgctttaccaacgtgtacgccgacagctttgtgatcagaggcgacgaggtgagacagattgctcctggacagaccggcaacattgccgattacaattacaagctgcctgatgatttcaccggctgtgtgatcgcctggaacagcaacaacctggacagcaaggtgggcggcaactataattacctgtatagactgttcagaaaaagcaacctgaagcctttcgagagagacatctccaccgaaatttaccaagccggatccacaccttgcaacggcgtgaagggcttcaattgttacttccctctgcaaagctacggctttcagcctacatacggagtgggctaccaaccctacagagtggtcgtgctgagcttcgagctgctccacgcccctgccaccgtgtgcggccctaaaaagagcaccaacctggtgaagaataagagagtgcaacccacagagagcattgtgagattccctaacatcacaaatctgtgccctttcggcgaggtgttcaacgccacaagattcgctagcgtgtacgcttggaacagaaagagaatcagcaactgtgtggctgactacagcgtgctctacaacagcgcttccttcagcacctttaaatgctacggcgtgagccccacaaagctcaacgacctgtgcttcaccaacgtctacgccgacagcttcgtgattagaggagacgaagtgagacagatcgctcctggacagaccggcaatatcgccgactacaactacaaactgcctgacgacttcaccggctgcgtgatcgcctggaacagcaataatctcgacagcaaggtcggcggcaactacaattacctgtatagactgtttagaaagagcaacctgaagcccttcgagagagacatctccaccgagatctaccaagccggctccacaccttgcaacggcgtgaagggcttcaactgctacttccctctgcagagctacggcttccaacctacctacggagtgggctatcagccttatagagtggtcgtgctgagcttcgaactgctgcacgcccccgccaccgtgtgcggccccaaaaagagcacaaacctggtgaagaacaagtga

SEQ ID NO: 14 - DNA, 1977 bp, Artificial Sequence
  SARS-CoV-2
atgagagtgcagcctaccgagagcatcgtgagattccctaacatcacaaacctctgcccttttggagaggtcttcaacgccacaagattcgcctccgtgtatgcctggaacagaaagagaatcagcaattgcgtggctgactacagcgtgctgtacaactccgctagctttagcaccttcaagtgctacggcgtgagccctaccaaactcaacgatctgtgctttaccaacgtctatgccgacagctttgtgatcagaggcgacgaggtcagacaaatcgcccccggacagaccggcaagatcgccgactacaactataaactgcctgacgacttcaccggctgcgtgatcgcctggaacagcaacaacctggactccaaggtgggcggcaactacaactacctgtacagactcttcagaaagagcaacctgaaacctttcgaaagagacatcagcaccgaaatctaccaagccggcagcaccccttgcaacggcgtggagggctttaattgctactttcctctgcaaagctacggctttcagcccaccaacggagtgggctatcagccttatagagtcgtcgtgctgagctttgagctgctgcacgcccctgccaccgtctgcggccctaaaaagagcacaaatctggtcaagaacaagagagtgcagcctaccgagagcattgtgagatttcctaacatcaccaatctgtgccctttcggcgaggtgttcaatgccacaagatttgctagcgtctacgcctggaacagaaaaagaatcagcaactgcgtggccgactacagcgtgctgtacaactccgctagcttcagcacctttaagtgctacggcgtcagccctaccaaactgaacgacctctgctttaccaacgtgtatgccgacagcttcgtgatcagaggagacgaggtgagacagatcgcccctggacagaccggcaacatcgccgattacaattacaaactgcccgacgacttcaccggctgtgtgattgcttggaactccaacaacctcgacagcaaggtgggcggcaactacaactacctgtatagactgttcagaaagagcaacctgaagcctttcgagagagatatcagcaccgagatctaccaagccggcagcacaccttgtaacggcgtgaaaggcttcaattgttatttccccctgcagagctacggctttcagcctacctatggcgtgggataccaaccttacagagtggtcgtgctgagcttcgaactgctgcacgcccctgccaccgtgtgcggccccaaaaagagcaccaatctggtgaagaacaaaagagtgcaacctaccgagagcatcgtgagattccccaatattacaaacctgtgcccctttggcgaggtgttcaacgccacaagattcgctagcgtgtacgcctggaacagaaagagaatcagcaactgcgtggctgactacagcgtgctgtacaacagcgcctccttcagcacattcaagtgctacggagtcagccctaccaagctgaatgatctgtgtttcacaaacgtgtacgccgacagcttcgtgatcagaggcgacgaagtgagacagattgcccctggacagaccggcaacatcgccgactacaattacaagctgcctgacgatttcaccggctgcgtcatcgcctggaacagcaacaacctggactccaaagtgggcggcaactacaattacctgtatagactgttcagaaagtccaatctcaagcccttcgagagagacatcagcaccgagatttatcaagccggcagcaccccttgcaatggagtgaaaggcttcaactgctacttccctctgcagagctacggatttcagcctacctacggagtgggctatcagccttacagagtggtcgtgctgagctttgagctgctgcacgctcctgccaccgtgtgcggccctaaaaagagcaccaacctggtgaagaacaagtga

SEQ ID NO: 15 - DNA, 1977 bp, Artificial Sequence
  SARS-CoV-2
atgagagtgcagcctacagagagcattgtgagattccctaacatcacaaacctgtgccctttcggcgaggtgttcaacgccacaagattcgctagcgtgtacgcctggaacagaaaaagaattagcaattgcgtcgccgattacagcgtgctgtataacagcgcttccttcagcaccttcaagtgctacggcgtcagccctacaaagctgaacgacctgtgcttcacaaacgtgtatgccgacagcttcgtgatcagaggcgacgaggtgaggcaaatcgctcctggccaaaccggcaacatcgccgactacaattacaagctgcctgatgacttcaccggatgcgtgattgcctggaacagcaacaacctggacagcaaagtcggaggaaactacaactacctgtacagactgttcagaaagagcaacctgaagcctttcgagagagacatcagcaccgagatctaccaagccggctccacaccttgcaacggcgtgaagggattcaactgctacttccctctgcagagctacggctttcagcccacctacggcgtgggctatcagccttacagagtggtcgtcctgagctttgagctgctccacgcccctgccaccgtctgcggccccaaaaaaagcaccaatctggtgaagaacaagagggtgcagcctacagagagcatcgtgagattccctaacattaccaacctgtgccctttcggagaagtgtttaacgccacaagattcgcctccgtctacgcttggaatagaaagaggatcagcaactgcgtggccgactacagcgtgctgtacaattccgcctccttcagcaccttcaagtgttacggcgtgagccctaccaagctgaacgacctgtgcttcaccaacgtgtacgccgatagcttcgtgattagaggcgacgaggtgaggcagatcgcccctggacaaaccggcaagattgctgactacaattacaagctgcctgacgacttcaccggctgcgtgatcgcctggaacagcaacaacctggacagcaaggtcggaggcaattacaattacctctatagactgttcagaaaaagcaatctcaagcctttcgaaagagacattagcaccgagatctaccaagccggcagcaccccctgcaacggcgtggaaggattcaactgctacttccccctgcagagctacggctttcagcccaccaacggagtgggctatcaaccctacagagtggtcgtgctctccttcgagctgctgcatgcccctgccacagtgtgcggacctaaaaagtccaccaacctcgtgaagaacaagagagtgcagcctacagagagcatcgtgaggttccccaacatcaccaacctgtgccctttcggcgaggtgtttaacgctacaagattcgctagcgtgtacgcttggaacagaaagagaatctccaattgcgtggccgactacagcgtgctgtacaacagcgctagcttcagcaccttcaagtgctacggagtgtcccctaccaagctgaacgacctgtgcttcaccaacgtgtacgccgatagcttcgtgattagaggagacgaagtgagacagattgctcctggacagaccggcaacatcgccgactacaattacaagctgcctgatgactttaccggctgtgtgattgcctggaacagcaacaacctggacagcaaggtgggcggcaactacaactatctgtacagactcttcagaaagagcaatctgaagccttttgaaagggacatcagcaccgagatctatcaagccggcagcaccccttgcaacggagtcaagggcttcaactgctactttcctctgcagagctatggctttcagcctacctacggcgtcggatatcagccttacagagtcgtggtgctgagcttcgagctgctccacgcccctgccaccgtctgcggccctaagaaaagcaccaacctggtcaagaacaaatga

SEQ ID NO: 16 - DNA, 1977 bp, Artificial Sequence
  SARS-CoV-2
atgagggtgcagcccacagagagcatcgtcagattccctaacatcacaaacctgtgtccttttggcgaggtcttcaacgccacaagattcgctagcgtgtacgcctggaatagaaaaaggattagcaactgtgtggctgactacagcgtcctgtataacagcgcttccttttccacctttaagtgttatggcgtgtcccccacaaagctgaacgacctgtgttttaccaatgtgtacgccgacagcttcgtcatcagaggagacgaggtgagacaaatcgcccctggacagaccggcaacatcgccgactacaattataagctgcctgacgactttaccggctgcgtgattgcttggaactccaacaacctggatagcaaggtgggaggaaactacaactacctctacagactcttcagaaagagcaatctgaagcctttcgagagagatatctccacagagatctatcaagccggcagcaccccttgtaacggagtgaagggattcaattgttatttccctctgcaaagctatggatttcagcctacctacggagtgggatatcagccctatagagtggtcgtgctgagcttcgagctcctgcacgcccctgctacagtctgtggccctaagaagtccacaaacctcgtgaagaacaagagagtgcagcctaccgagagcatcgtgaggttccctaacattaccaacctctgtccttttggagaagtctttaatgccacaagatttgctagcgtgtacgcttggaatagaaagaggatttccaattgcgtggccgactactccgtcctgtacaacagcgctagcttcagcaccttcaaatgctacggagtgagccctaccaagctcaacgacctgtgctttaccaacgtgtacgctgacagcttcgtcattaggggagacgaggtgaggcagatcgctcctggacagaccggcaacatcgccgactacaactacaagctgcccgacgatttcaccggctgcgtcattgcctggaatagcaacaatctggacagcaaggtgggaggaaactataattacctgtacagactgtttaggaaaagcaacctcaaacccttcgaaagagacattagcacagagatctaccaagccggctccaccccctgcaatggcgtgaagggatttaattgttacttccccctgcagagctatggctttcaacctacctacggcgtgggctatcagccctatagggtggtggtcctcagctttgaactcctgcacgctcctgccaccgtgtgcggccccaaaaagagcaccaatctggtcaagaacaagagagtgcagcccacagagtccatcgtgagatttcctaatattaccaacctgtgccctttcggagaggtgtttaatgctacaagatttgctagcgtctatgcctggaacagaaagagaatcagcaactgcgtggccgattacagcgtcctgtacaatagcgcttccttctccacctttaaatgctacggcgtgagccccaccaagctgaatgacctctgtttcaccaacgtgtatgccgactcctttgtgattagaggagatgaggtgagacagatcgcccctggacaaaccggcaacattgccgattacaattacaagctgcctgatgacttcaccggctgtgtgattgcctggaacagcaataacctggacagcaaggtgggcggcaattataactacctgtatagactgttcagaaagagcaacctgaagccttttgagagagacatcagcaccgagatttaccaagccggctccaccccttgcaacggcgtgaagggcttcaattgctacttccctctgcagtcctacggctttcagcctacatacggcgtgggatatcagccttatagagtcgtggtgctcagcttcgagctgctgcacgcccccgctacagtgtgtggacctaaaaagtccaccaatctcgtcaagaataagtga

SEQ ID NO: 17 - DNA, 1977 bp, Artificial Sequence
  SARS-CoV-2
atgagagtgcagcctaccgagagcatcgtgagattccctaacatcaccaacctctgccctttcggagaggtcttcaacgccacaagatttgctagcgtgtacgcctggaacagaaaaagaatctccaactgcgtggccgactacagcgtcctgtacaatagcgctagcttcagcaccttcaagtgctacggcgtctcccctacaaagctgaatgacctgtgcttcaccaacgtgtatgccgacagcttcgtgattagaggcgatgaggtgaggcagatcgcccctggacaaaccggcaacatcgccgattataactacaagctgcctgacgacttcaccggctgcgtgatcgcctggaacagcaacaacctggacagcaaggtcggcggcaactacaactacctctacagactgttcagaaagtccaacctgaagcctttcgagagggacatcagcaccgagatttaccaagccggcagcaccccttgcaacggcgtgaagggatttaactgctatttccctctgcagagctacggcttccaacctacctatggagtgggctaccaaccctacagagtggtggtcctgagcttcgaactgctgcacgcccctgccaccgtgtgtggccctaaaaagagcaccaatctcgtgaaaaacaagagagtgcagcctacagaaagcattgtgagatttcctaatatcaccaacctgtgccctttcggcgaggtgttcaacgctacaagattcgctagcgtgtacgcctggaatagaaagagaatcagcaactgcgtggccgactacagcgtgctgtacaacagcgcttcctttagcaccttcaagtgttacggcgtgagccccaccaagctcaacgacctgtgcttcacaaacgtgtacgctgacagcttcgtgatcagaggcgatgaggtgagacagatcgcccctggccaaaccggcaacatcgccgattacaactataagctccctgatgacttcaccggctgcgtgatcgcttggaacagcaacaacctggacagcaaggtgggcggcaactacaactacctctatagactgttcagaaaatccaacctgaagcctttcgagagagacatctccaccgagatttatcaagccggaagcaccccctgcaatggcgtgaagggcttcaactgctacttccctctccaatcctacggcttccaacctacctacggagtgggctatcagccctacagagtggtcgtgctgagcttcgagctcctgcacgcccctgccaccgtgtgcggccctaaaaagagcaccaacctggtcaaaaataagagagtgcagcccaccgagagcatcgtgagattccctaatatcaccaacctgtgcccttttggagaggtgttcaacgccacaagattcgctagcgtgtacgcctggaatagaaagaggatcagcaactgcgtggccgactacagcgtgctctacaacagcgcctccttcagcacctttaagtgttacggcgtctcccctaccaagctgaacgatctctgctttaccaacgtgtacgccgacagctttgtgatcagaggcgacgaagtgaggcaaatcgctcctggacagaccggaaacatcgccgactacaactacaagctgcctgacgacttcaccggctgcgtcatcgcctggaatagcaacaacctggatagcaaagtcggaggcaactacaactacctgtacagactcttcagaaagagcaacctcaaacctttcgagagagacatcagcacagagatctaccaagccggcagcaccccttgcaacggagtcaagggcttcaactgctactttcctctgcagagctacggctttcagcctacctacggcgtcggctatcagccttacagagtggtcgtgctcagcttcgaactcctgcacgcccccgccaccgtgtgcggacctaaaaagagcaccaatctggtgaagaacaaatga

SEQ ID NO: 18 - protein, 658 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

SEQ ID NO: 19 - protein, 439 aa, Artificial Sequence
  SARS-CoV-2
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK